(19) United States
(12) Patent Application Publication
(10) Pub. No.: US 2015/0376673 A1
(43) Pub. Date: Dec. 31, 2015

(54) Cell-Free Translation System

(71) Applicants: INSTITUT NATIONAL DE LA SANTE ET DE LA RECHERCHE MEDICALE (INSERM), Paris (FR); UNIVERSITE CLAUDE BERNARD LYON 1, Villeurbanne (FR); ECOLE NORMALE SUPERIEURE DE LYON, Lyon (FR)

(72) Inventors: Laurent Balvay, Lyon Cedex 07 (FR); Didier Decimo, Lyon Cedex 07 (FR); Baptiste Panthu, Lyon Cedex 07 (FR); Theophile Ohlmann, Lyon Cedex 07 (FR)

(21) Appl. No.: 14/766,636

(22) PCT Filed: Feb. 6, 2014
(86) PCT No.: PCT/EP2014/052369

§ 371 Date: Aug. 7, 2015

(30) Foreign Application Priority Data
Feb. 8, 2013 (EP) 13305158.1

(51) Int. Cl. C12P 21/00; C12N 9/00

(52) U.S. Cl. CPC C12P 21/00; C12N 9/93; C12Y 601/01

(57) ABSTRACT

The present invention relates to a new cell-free translation system. In particular, the invention relates to a cell-free reaction system for translating in vitro a RNA into a protein, said reaction system comprising a ribosome-depleted red blood cell lysate and ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes. The invention also pertains to a method for translating in vitro a ribonucleic acid template into an amino acid sequence of interest using the cell-free reaction system of the invention. The invention also relates to the use of (i) a ribosome-depleted red blood cell lysate, and (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes, for producing a cell-free translation system.

The present invention relates to a new cell-free translation system.

In particular, the invention relates to a cell-free reaction system for translating in vitro a RNA into a protein, said reaction system comprising a ribosome-depleted red blood cell lysate and ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

The invention also pertains to a method for translating in vitro a ribonucleic acid template into an amino acid sequence of interest using the cell-free reaction system of the invention.

The invention also relates to the use of (i) a ribosome-depleted red blood cell lysate, and (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes, for producing a cell-free translation system.

### BACKGROUND OF THE INVENTION

Cell free protein synthesis systems (hereinafter abbreviated as “CFPS”), also called cell-free translation system, have been used for decades to express proteins from ectopically added nucleic acids that could be either mRNAs or cDNAs (for review, see Carlson et al, Biotechnol Adv., 30(5): 1185-1194, 2012).

CFPS are excellent tools to rapidly and efficiently produce proteins, for instance proteins with a pharmaceutical interest such as vaccine components and cytokines (Kanter et al. Blood, 109(8): 3393-3399, 2007; Yang et al. Biotechnol Prog., 20(6): 1689-1696, 2004; Yang et al. Biotechnol Bioeng, 89(5): 503-511, 2005; Zawada et al. Biotechnol Bioeng, 108(7): 1570-1578, 2011). Further, CFPS are routinely exploited to perform high throughput production of protein libraries (Goshima et al. Nat Methods, 5(12): 1011-1017, 2008). In vitro translational assays represent a good alternative for directly sampling and screening molecules due to the absence of cell wall. In addition, expression of proteins in vitro enables synthesis of toxic proteins that can not be produced in live cells.

CFPS are prepared from crude cell extracts containing all necessary components for energy generation and the complete apparatus required for translation of RNA (e.g. ribosomes, tRNAs and aminoacyl-tRNA synthetases, initiation, elongation and termination factors, chaperones, amino acids etc. . . . ). However, to ensure efficient translation, usually cell extracts are supplemented with amino acids, energy sources (e.g. ATP, GTP), energy regenerating systems (e.g. creatine phosphate and creatine phosphokinase for eukaryotic systems, and phosphoenol pyruvate and pyruvate kinase for the E. coli lysate), and other co-factors (Mg²⁺, K⁺, etc.). An advantage of CFPS is that the concentration of the endogenous components can be manipulated by chemicals, enzymes, or modified by the addition of recombinant proteins (Ohlmann et al. EMBO J., 16(4): 844-855, 1997; Ohlmann et al. J Mol Biol., 318(1): 9-20, 2002; Ziegler et al. Virology, 213(2): 549-557, 1995; Ziegler et al. J Virol., 69(6): 3465-3474, 1995). In addition, the short incubation time and the good level of standardization of commercially available CFPS render experimental procedures very easy to use and reproduce. All these features make in vitro systems very powerful tools for research and protein production.

While, in theory, preparation of translation competent cell free extracts from any type of cells could seem easy, actually that is not the case. Indeed, only a few cell-free active systems have been developed in the last 3 decades. The most frequently used cell-free translation systems consist of extracts from rabbit reticulocytes, insect cells, wheat germ and Escherichia coli. (Carlson et al, Biotechnol Adv., 30(5): 1185-1194, 2012). Whilst the E. coli, wheat germ and insect cells are excellent tools to produce large amounts of a given protein, they are not adequate for studies on the pathway of mammalian translational control.

For this reason, mammalian translational studies have been generally conducted with the rabbit reticulocyte lysate which was developed in 1974 by Hunt and Jackson (Hamatol Bluttransfus, 14: 300-307) and optimized for protein production by disposing of the endogenous mRNAs with the calcium activated S7 microccocale nuclease (Pelham & Jackson. Eur J Biochem., 67(1): 247-256, 1976). Since then, both the untreated (hereinafter abbreviated as “URRL”) or nuclease treated rabbit reticulocyte lysate (hereinafter abbreviated as “RRL”) have been successfully commercialized and widely used by the scientific community.

However, a major concern with the reticulocyte lysate is that it does not recapitulate some important translation characteristics that are found in a cellular environment. One of them concerns the lack of cap- and poly(A) dependence which is a critical determinant in translational control (Beilharz et al. Prog Mol Subcell Biol., 50: 99-112, 2010; Lemay et al. RNA Biol., 7(3): 291-295, 2010; Tomek & Wollenhaupt, Anim Reprod Sci., 134(1-2): 2-8, 2012).

It has been shown that partial depletion of ribosomes and ribosome-associated factors can recreate in the reticulocyte lysate the selective advantage conferred by addition of the cap and poly(A) tail to the mRNA, but at the same time, such a depletion has an impact on the yield of proteins produced (Borman et al. Nucleic Acids Res., 28(21): 4068-4075, 2000; Michel et al. J Biol Chem., 275(41): 32268-32276, 2000). Likewise, it has been shown that the untreated RRL could recreate cap/poly(A) synergy but the presence of endogenous globin and lipoxygenase mRNAs may interfere with RNA and protein expression (Soto Rifo et al. Nucleic Acids Res., 35(18): e121, 2007).

Finally, the physiological relevance of the RRL has often been criticized for the study of human genes or viral RNAs that infect humans as it may not contain all factors required for their expression. Along this line, it is noteworthy that many IRES-driven mRNAs of cellular and viral origins are not, or poorly, translated in the rabbit reticulocyte lysate (Borman et al. Nucleic Acids Res., 25: 925-932, 1995; Borman et al. Nucleic Acids Res., 25(5): 925-932, 1997; Stoneley et al. Nucleic Acids Res., 28(3): 687-694, 2000; Stoneley & Willis, Oncogene, 23(18): 3200-3207, 2004).

To address these issues, several in vitro translation systems based on extracts from mammalian cells have been developed over the last years (Bergamini et al. Rna., 6(12): 1781-1790, 2000; Svitkin & Sonenberg, Methods Mol Biol., 257: 155-170, 2004; Thoma et al. Methods Mol Biol., 257: 171-180, 2004; Witherell, Curr Protoc Cell Biol., Chapter 11: Unit 11 18, 2001). Recently, the company Pierce Biotechnology Inc. has made a HeLa based translation system commercially available. Whilst all these systems are faithful to recreate a competitive cellular environment, they are usually tedious to make and quite inefficient in terms of the yield of protein produced. Although they are suitable to use with sensitive reporter genes such as luciferases whose activity can be determined enzymatically it is often very difficult to visualize the synthesis of a given gene by the readout of [35S]-methionine incorporation. This is a major drawback as it is often necessary to observe the translation product of a given gene in order to ensure that it is not degraded or truncated or to visualize any isoforms that can be produced by alternative translation initiation or internal initiation as it was shown for HIV-1, HIV-2 (Balvay et al. Nat Rev Microbiol., 5(2): 128-140, 2007; Balvay et al. Biochim Biophys Acta, 2009; de Breyne et al. Virus Res, S0168-1702(12)00376-0, 2012; Herbreteau et al. Nat Struct Mol Biol., 12(11): 1001-1007, 2005).

To date, neither of the existing in vitro translation system can combine a high expression yield with the recapitulation of the cellular physiological environment (i.e. features of translational control that are only found in a cellular competitive environment).

Therefore, it is of particular interest to develop a new cell-free translation system which does not suffer from the drawbacks of the in vitro translation systems of the prior art, i.e. a system that both allows a high protein production and optimally mimics the cellular physiological environment.

### DESCRIPTION OF THE INVENTION

In an attempt to develop a mammalian in vitro translation system that could combine efficient protein production with features of translational control usually found in a cellular competitive environment, the inventors have for the first time designed a highly adaptable in vitro system which relies on mixing i) components obtained from a red blood cell lysate (for instance a reticulocyte lysate, and in particular a rabbit reticulocyte lysate) depleted from its ribosomes, with ii) ribosomes that have been purified from eukaryotic cultured cells, in particular mammalian cells such as HeLa, Jurkat, BHK, mouse stem cells, undifferentiated myoblasts and differentiated myotubes.

Such a reconstituted in vitro lysate retains the high efficiency of the parental RRL and recapitulates translational characteristics observed in cells from which the ribosomes have been isolated.

Interestingly, the inventors have shown that addition of ribosomes from a given cell type is sufficient to confer the translational characteristics found in living cells. As such, the inventors recapitulated cap/poly(A) synergy, the selective advantage of IRES-driven translation and, in particular, cellular tropism that is observed in some cell types and with some specialized mRNAs. For instance, the inventors have reproduced translation stimulation of mRNAs that are activated by cell differentiation.

They have further shown that the system of the invention can also be used for the preparation of an in vitro translational assay in which endogenous proteins which are normally associated to the ribosomal pellet has been depleted by RNA interference.

Another interesting characteristic of this system is that ribosomes can be obtained from eukaryotic cells previously transfected with a DNA construct (for instance a plasmid comprising a cDNA) coding for a protein of interest, so that transcription of the DNA into mRNA occurs in cellulo with no need to go through an in vitro transcription procedure. In this embodiment, since the mRNAs are in contact with the ribosomes in the cells, the ribosomes are isolated together with the mRNAs. The advantage of this embodiment of the in vitro translation system of the invention is that it skips the in vitro transcription step and allows studying translation of transcripts that have been synthesized and processed in their native environment.

Still another characteristic of this system is that ribosomes can be obtained from eukaryotic cells infected with a virus, enabling studying translation of viral transcripts that have been synthesized and processed in the host cell. Ribosomes can also be obtained from eukaryotic cells infected with viruses, bacteria or protozoan, enabling studying the effect of these infections on the expression of genes from said eukaryotic cells.

Ribosomes can also be obtained from eukaryotic cells derived from healthy or pathogenic organs or tissues, enabling studying translation of transcripts that have been synthesized and processed in these specific organs and tissues.

The inventors have also shown that the in vitro translation system of the invention is efficient even with minute amount of RNA. Indeed, as few as 0.14 fmol (i.e. 0.14×10⁻¹⁵ mol) allows an efficient translation. In contrast, with other in vitro translation systems based on extracts from mammalian cells, in particular that marketed by Pierce Biotechnology Inc., no translation is detected when 0.14 fmol of RNA is used in the translational assay.

Accordingly, another characteristic of this system is that it enables analysing the actively translated transcriptome of eukaryotic cells of interest from RNAs, produced in said eukaryotic cells of interest and isolated together with the ribosomes from said eukaryotic cells of interest.

### SUMMARY OF THE INVENTION

In its broadest aspect, the invention relates to a new cell-free translation system comprising a ribosome-depleted red blood cell lysate and ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

The invention also relates to methods using this new cell-free translation system for producing a protein of interest, as well as the use of a ribosome-depleted red blood cell lysate and ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease rabbit untreated reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes, for producing an in vitro translation system.

The invention also concerns an in vitro method for analysing an actively translated transcriptome of eukaryotic cells of interest comprising:

a) isolating ribosomes from eukaryotic cells of interest under conditions that allow RNAs produced in the eukaryotic cells of interest to be isolated together with the ribosomes;

b) incubating a translation system comprising (i) a ribosome-depleted red blood cells lysate and (ii) ribosomes and RNAs isolated from the eukaryotic cells of interest in step a), for a time sufficient to achieve translation of the RNAs, isolated with the ribosomes in step a), into the corresponding amino acid sequences,

with the proviso that (1) when the ribosome-depleted red blood cell lysate is obtained from a nuclease untreated rabbit reticulocyte lysate, the eukaryotic cells of interest from which ribosomes are isolated are not nuclease rabbit untreated reticulocytes, and (2) when the ribosome-depleted red blood cell lysate is obtained from a nuclease treated rabbit reticulocyte lysate, the eukaryotic cells of interest from which ribosomes are isolated are not nuclease treated rabbit reticulocytes; and

c) identifying and optionally quantifying the amino acid sequences obtained in step b).

Further, the invention relates to a kit for translating in vitro a RNA into a protein of interest comprising a ribosome-depleted red blood cells lysate and ribosomes isolated from eukaryotic cells.

### DETAILED DESCRIPTION OF THE INVENTION

These and other objects, features and advantages of the invention will be disclosed in the following detailed description.

A Method for Translating In Vitro a Ribonucleic Acid Template into an Amino Acid Sequence of Interest

According to a first aspect, the invention relates to a method for translating in vitro a ribonucleic acid template into an amino acid sequence of interest, the method using a translation reaction mixture comprising: (i) a ribosome-depleted red blood cells lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

In an embodiment, the present invention provides a method for translating in vitro a ribonucleic acid template into an amino acid sequence of interest, the method comprising:

pre-a) optionally preparing a translation reaction mixture comprising: (i) a ribosome-depleted red blood cells lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes;

a) to form a translation system, contacting the translation reaction mixture as defined in step (pre-a) with:

(i) the ribonucleic acid template (RNA); or

(ii) a transcription reaction mixture comprising (i) a DNA encoding the amino acid sequence of interest, and (ii) the necessary element for transcribing said DNA into the ribonucleic acid template;

b) incubating the translation system of (a) for a time sufficient to achieve translation of the ribonucleic acid template into the amino acid sequence of interest.

More particularly, in an embodiment, the present invention provides a method for translating in vitro a ribonucleic acid template into an amino acid sequence of interest, the method comprising:

a) to form a translation system, contacting a translation mixture comprising: (i) a ribosome-depleted red blood cells lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes, with:

(i) the ribonucleic acid template (RNA); or

(ii) a transcription reaction mixture comprising (i) a DNA encoding the amino acid sequence of interest, and (ii) the necessary element for transcribing said DNA into the ribonucleic acid template;

and

b) incubating the translation system of (a) for a time sufficient to achieve translation of the ribonucleic acid template into the amino acid sequence of interest.

In another embodiment, the present invention provides a method for translating in vitro a ribonucleic acid template into an amino acid sequence of interest, the method comprising:

pre-a) preparing a translation reaction mixture comprising: (i) a ribosome-depleted red blood cells lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes;

a) to form a translation system, contacting the translation reaction mixture as defined in step (pre-a) with:

(i) the ribonucleic acid template (RNA); or

(ii) a transcription reaction mixture comprising (i) a DNA encoding the amino acid sequence of interest, and (ii) the necessary element for transcribing said DNA into the ribonucleic acid template;

b) incubating the translation system of (a) for a time sufficient to achieve translation of the ribonucleic acid template into the amino acid sequence of interest.

Typically, step (b) is conducted during at least 15 minutes, preferably at least 30 minutes, and by order of preference at least 60 minutes, 75 minutes, 90 minutes, 120 minutes, 150 minutes. Further, step (b) is preferably conducted between about 30° C. and about 37° C., and more preferably at 30° C.

The ribonucleic acid template used in step (a) (i) may be readily produced by the one skilled in the art according to known methods. For instance, the RNA template may be produced by chemical synthesis or by in vitro transcription. It can also be a purified native template.

When the RNA template must have a poly(A) tail, the poly (A) tail of the RNA template can be encoded in a DNA coding sequence which can be transcribed to generate an RNA template with a poly (A) tail of defined length. An alternative method of generating the poly (A) tail is the use of a poly (A) polymerase in an in vitro reaction to add the tail to the template as a posttranscriptional modification.

A 5′ cap portion may be added co-transcriptionally to the RNA template by the RNA polymerase according to protocols well known to one skilled in the art. If the template is a purified native RNA template, the cap structure will already be in place.

In another embodiment, the ribosomes used to carried out the method are obtained from eukaryotic cells previously transfected, with a DNA (the DNA may be, for instance, a cDNA or a gene) coding for a protein of interest. Since the transcription occurs in cellulo and the translation begins within the cells, the mRNAs are in contact with the ribosomes. Thus, the ribosomes are isolated together with the mRNAs.

Therefore, in an embodiment, the invention concerns a method for translating in vitro a ribonucleic acid template into an amino acid sequence of interest, the method comprising:

pre-a) transfecting a DNA encoding the amino acid sequence of interest into eukaryotic cells, with proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes;

a) after a time sufficient to allow the eukaryotic transfected cells to achieve RNA transcription and to begin translation, isolating ribosomes from the transfected cells of step (pre-a) under conditions that allow the RNA produced from the DNA encoding the amino acid sequence of interest to be isolated together with the ribosomes; and

b) incubating a translation system comprising (i) a ribosome-depleted red blood cells lysate, (ii) ribosomes isolated from the eukaryotic cells in step (a) for a time sufficient to achieve translation of the ribonucleic acid template into the amino acid sequence of interest.

To allow the eukaryotic transfected cells to achieve RNA transcription and to begin translation, the cells are generally cultured during at least between 12 h and 48 h, preferably 36 hours after transfection.

To isolate the RNA linked to the ribosomes, it is better to add RNAse inhibitor during cell lysis then after ribosomal fraction resuspension (however if buffer are prepared in RNase free conditions and all ribosomal purification step are carried out at 4° C., RNase inhibitors are not inevitably required). Methods to isolate mRNA linked to the ribosomes are well known to the one skilled in the art.

Typically, step (b) is conducted preferably at about 30° C., during at least 15 minutes, preferably at least 30 minutes, and by order of preference at least 60 minutes, 75 minutes, 90 minutes, 120 minutes, 150 minutes. Further, step (b) is preferably conducted between about 30° C. and about 37° C., and more preferably at 30° C.

The different embodiments of the method for translating in vitro a ribonucleic acid template into an amino acid sequence of interest according to the present invention can be performed by using a batch system in a conventional manner. Alternatively, they may be carried out by using various already known or usual methods such as a flow method, wherein materials including the components of the translation reaction mixture are continuously supplied or the reaction product is occasionally withdrawn.

Use of a Mixture Comprising Ribosome-Depleted Red Blood Cell Lysate and Ribosomes Isolated from Eukaryotic Cells

According to a second aspect, the invention relates to the use of a translation reaction mixture comprising (i) a ribosome-depleted red blood cell lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes, for in vitro producing a peptide.

According to a third aspect, the invention relates to the use of a ribosome-depleted red blood cell lysate and ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes, for producing translation reaction mixture.

A Cell-Free Translation Reaction System

According to a fourth aspect, the invention relates to a cell-free translation reaction system for translating in vitro a RNA into a protein comprising: (i) a ribosome-depleted red blood cell lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

A Method for Analysing an Actively Translated Transcriptome

According to a fifth aspect, the invention relates to an in vitro method for analyzing an actively translated transcriptome of eukaryotic cells of interest comprising:

a) isolating ribosomes from eukaryotic cells of interest under conditions that allow RNAs produced in the eukaryotic cells of interest to be isolated together with the ribosomes;

b) incubating a translation system comprising (i) a ribosome-depleted red blood cells lysate and (ii) ribosomes and RNAs isolated from the eukaryotic cells of interest in step a), for a time sufficient to achieve translation of the RNAs, isolated with the ribosomes in step a), into the corresponding amino acid sequences,

with the proviso that (1) when the ribosome-depleted red blood cell lysate is obtained from a nuclease untreated rabbit reticulocyte lysate, the eukaryotic cells of interest from which ribosomes are isolated are not nuclease rabbit untreated reticulocytes, and (2) when the ribosome-depleted red blood cell lysate is obtained from a nuclease treated rabbit reticulocyte lysate, the eukaryotic cells of interest from which ribosomes are isolated are not nuclease treated rabbit reticulocytes; and

c) identifying and optionally quantifying the amino acid sequences obtained in step b).

As used herein, the term “actively translated transcriptome” refers to the set of mRNAs in a cell which are associated with ribosomes and are therefore in the process of being translated.

Step c) of identifying and optionally quantifying the amino acid sequences obtained in step b) may be carried out by any technique well-known from the skilled person such as mass spectrometry, in particular liquid chromatography tandem mass spectrometry (LC/MS/MS); peptide mass fingerprinting; or at least partial amino acid sequencing, in particular Edman degradation. Preferably, the amino acid sequences are identified and optionally quantified in step c) by mass spectrometry.

The translation system incubated in step b) of the method of analysis preferably comprises all necessary components required for achieving proper translation of an RNA into an amino acid sequence. Such necessary components are well known from the skilled person, and include typically tRNAs and aminoacyl-tRNA synthetases, initiation, elongation and termination factors, chaperones, foldases, amino acids (e.g. natural, unnatural, standard and/or non-standard amino acids), energy sources (e.g. nucleotide triphosphate such as ATP, GTP), energy regenerating systems (e.g. creatine phosphate and creatine phosphokinase, myokinase), and salts (Mg²⁺, K⁺, etc.). Accordingly, in a preferred embodiment, the translation system incubated in step b) further comprises at least one element selected from the group consisting of at least a tRNA, at least an aminoacyl-tRNA synthetase, at least an initiation factor, at least an elongation factor, at least a termination factor, at least a chaperone, at least a foldase, at least an amino acid, at least a labelled amino acid, at least an energy source, at least an energy regenerating system and salts.

In a particularly preferred embodiment, the translation system incubated in step b) further comprises at least a labeled amino acid, such as a radiolabeled amino acid ([³⁵S]-methionine, [³⁵S]-cystéine, [³H]/[¹⁴C]/[¹⁵N]-amino-acids . . . ), a photoreactive amino acid (for instance diazirine-based amino acid analogs), or a biotinylated amino acid. More preferably, the translation system incubated in step b) further comprises radiolabeled amino acids, in particular [¹⁴C]-amino acids and/or [¹⁵N]-amino acids.

Advantageously, in a particular embodiment, the method of analysis according to the invention can use puromycin-associated nascent chain proteomics (PUNCH-P), as described in Aviner et al. (2013) Genes & Dev. 27:1834-1844, which is based on incorporation of biotinylated puromycin into newly synthesized proteins under cell-free conditions followed by streptavidin affinity incorporation and liquid chromatography-tandem mass spectrometry analysis.

Accordingly, in a preferred embodiment, the translation system incubated in step b) further comprises biotinylated puromycin, such as 5′ biotin-dC-puromycin 3′ as for example described in Starck et al. (2004) Chem. & Biol. 11:999-1008. In this particular embodiment, the method of the invention preferably further comprises a step b′) of capturing the puromycin-labelled amino acid sequences obtained in step b) on immobilized streptavidin. In this particular embodiment, the amino acid sequences captured in step b′) are preferably identified and optionally quantified in step c) by liquid chromatography-tandem mass spectrometry.

The above described methods of analysis of an actively translated transcriptome are particularly advantageously because they can be implemented in the form of high throughput methods.

Kits According to the Invention

According to a sixth aspect, the invention relates to kits that are useful in the above methods and use. Such kits comprise, in separate containers or in the same container, (i) a ribosome-depleted red blood cell lysate, and optionally (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

Optionally, the kits comprise, in separate containers or in the same container, at least one element chosen from the group consisting of:
at least a tRNA;
at least an aminoacyl-tRNA synthetase;
at least an initiation factor;
at least an elongation factors;
at least a termination factor;
at least a chaperone;
at least a foldase;
at least an amino acid (e.g. a natural, unnatural, standard and/or non-standard amino acid);
at least a labelled amino acid, for instance a radiolabelled amino acid;
at least an energy source (e.g. nucleotide triphosphate such as ATP, GTP);
at least an energy regenerating system (e.g. creatine phosphate and creatine phosphokinase, myokinase);
salts (Mg²⁺, K⁺, etc.);
a buffer solution (cell lysis buffer, ribosome resuspension buffer, reticulocyte supernatant used as translation buffer).
a pestle for ribosome pellet resuspension.

The kits according to the invention may further comprise, in separate containers or in the same container, at least a biotinylated puromycin and optionally at least an immobilized streptavidin.

The present invention also concerns a kit for analyzing an actively translated transcriptome of eukaryotic cells of interest comprising, in separate containers or in the same container:
a ribosome-depleted reticulocyte lysate, and
at least one element chosen from the group consisting of:
   at least a labelled amino acid,
   at least a biotinylated puromycin, and
   at least an immobilized streptavidin.

The kits according to the invention may also comprise a control sample comprising a given RNA template. The kits according to the invention may further comprise instructions for the use of said kit (i) in translating a RNA into an amino acid sequence, and/or (ii) in producing translation reaction mixture.

The kits may also include means for transcribing a DNA of interest into the corresponding RNA. These means include in particular a vector or a plasmid (wherein the DNA of interest can be cloned to be under control of a promoter), an appropriate RNA polymerase, rCTP, rATP, rUTP, rGTP and an appropriate buffer system. In this embodiment, the kits are suitable for performing a coupled transcription/translation reaction. Preferably, the means for transcribing a DNA of interest are not comprised in the container(s) that comprise(s) the ribosome-depleted reticulocyte lysate and the ribosomes isolated from eukaryotic cells.

In a first implementation of the first to sixth aspects of the invention according to any one of the embodiments disclosed, the eukaryotic cells from which ribosomes (ii) are isolated are not reticulocytes.

In a second implementation of the first to sixth aspects of the invention according to any one of the embodiments disclosed or according to the first implementation, the ribosome-depleted red blood cell lysate (i) is obtained from rabbit red blood cells, preferably from rabbit reticulocytes.

In a third implementation of the first to sixth aspects of the invention according to any one of the embodiments disclosed or according to the first or the second implementation, the eukaryotic cells from which ribosomes (ii) are isolated are human cells, preferably said human cells are not red blood cells.

In a fourth implementation of the first to sixth aspects of the invention according to any one of the embodiments disclosed or according to any one of the first to the third implementations, the ribosome-depleted red blood cell lysate (i) is obtained from a red blood cell lysate treated with a nuclease (e.g. a calcium activated S7 microccocale nuclease) to dispose of the endogenous mRNAs that are contained into the reticulocytes.

### DEFINITION

By “an amino acid sequence” is meant any type of natural and unnatural peptide, polypeptide or protein.

The term “natural” as used herein means amino acid sequences which comprise “standard” amino acids (i.e. Alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), and/or “non-standard” amino acids such as pyrrolysine, selenomethionine and selenocysteine (in specific conditions, selenocysteine is encoded by a UGA codon). The natural amino acids may be in the form of dextrogyre or levogyre optical isomers.

The term “unnatural” as used herein means amino acid sequences which have at least one modified natural amino acid, for instance a modified non-charged amino acid, a modified acidic amino acid, a modified basic amino acid, a non-alpha-amino acid, an amino acid having functional groups selected from the group consisting of nitro, amidine, hydroxylamine, quinone, aliphatic compounds, an amino acid residue such as p-fluorophenylalanine, p-nitrophenylalanine or homophenylalanine. The unnatural amino acids may be in the form of dextrogyre or levogyre optical isomers.

The expression “translation reaction mixture” refers to a composition which comprises all necessary components required for achieving proper translation of an RNA into an amino acid sequence.

All the components necessary for in vitro translation are well known to those skilled in the art, and are described by Soto et al. (Nucleic Acids Res., 35(18): e121, 2007) Pelham H R and Jackson R J (Eur. J. Biochem., 67(1): 247-256, 1976) and Rau M et al., Methods Mol. Biol., 77: 211-226, 1998). They include tRNAs and aminoacyl-tRNA synthetases, initiation, elongation and termination factors, chaperones, foldases, amino acids (e.g. natural, unnatural, standard and/or non-standard amino acids), energy sources (e.g. nucleotide triphosphate such as ATP, GTP), energy regenerating systems (e.g. creatine phosphate and creatine phosphokinase, myokinase), and salts (Mg²⁺, K⁺, etc.). The translation reaction mixture preferably comprises a buffer system (about pH 7.3).

Optionally, the translation reaction mixture may comprise labelled amino acids, for instance radiolabeled amino acids ([³⁵S]-methionine, [³⁵S]-cystéine, [³H]/[¹⁴C]/[¹⁵N]-amino-acids . . . ), photoreactive amino acids (for instance diazirine-based amino acid analogs), biotinylated amino acids, to help detection of the translated amino acid sequence.

The only source of ribosomes of the translation system of the invention is the ribosomal pellet isolated from eukaryotic cells.

Preferably, the eukaryotic cells used for obtaining the ribosomes (ii) are mammalian cells, for instance human cells, rabbit cells, rodent (e.g. mouse, hamster, guinea pig and rat) cells, horse cells, cow cells, dog cells, cat cells, goat cells, sheep cells etc. . . . . . In a preferred embodiment, the ribosomes are isolated from rodent cells or human cells.

The mammalian cells may be differentiated (mature) cells or undifferentiated such as adult or embryonic stem cells and progenitor cells.

The eukaryotic cells used for obtaining the ribosomes (ii) may be derived from eukaryotic cell lines or from organs or tissues. In particular, the eukaryotic cells used for obtaining the ribosomes (ii) may be derived from vertebrate, in particular mammalian, organs or tissues, for instance brain, heart, liver or lung.

The eukaryotic cells used for obtaining the ribosomes (ii) may be obtained from healthy or pathogenic eukaryotic cell lines, organs or tissues. In particular, the eukaryotic cells used for obtaining the ribosomes (ii) may be obtained from tumours, metastasis or biopsy samples. Said eukaryotic cells may also be eukaryotic cells infected with a virus, a bacterium or a protozoan.

Ribosomes can also be obtained from invertebrates such as drosophila and zebra fish cells and yeast.

The eukaryotic cells will be chosen regarding the wished cellular tropism and/or translational characteristics.

In the context of the invention, the term “isolated” or “purified” refers to biological molecules that are removed from their natural environment and are isolated or separated and are free from other components with which they are naturally associated.

Preferably, the ribosomes isolated from eukaryotic cells according to the invention are isolated from cytoplasmic extracts of these eukaryotic cells, more preferably from S10 supernatant extracts of these eukaryotic cells.

In particular, when the eukaryotic cells used for obtaining the ribosomes (ii) are derived from vertebrate organs or tissues, the ribosomes isolated from said eukaryotic cells are preferably isolated from whole extracts of said vertebrate organs or tissues.

Preferably, the ribosomes isolated from eukaryotic cells according to the invention are at least 70-95% pure ribosomes (optionally in association with RNAs and/or ribosome-associated proteins) by weight, preferably at least 75% pure ribosomes (optionally in association with RNAs and/or ribosome-associated proteins) by weight, still preferably at least 80%, 85% or 90% pure ribosomes (optionally in association with RNAs and/or ribosome-associated proteins) by weight, most preferably 98%, 99% or 100% pure ribosomes (optionally in association with RNAs and/or ribosome-associated proteins) by weight. Thus a cytoplasmic extract from eukaryotic cells including ribosomes, in particular a S10 supernatant extract of eukaryotic cells including ribodomes does not correspond to ribosomes isolated from encaryotic cells according to the invention since the extracts contain other eukaryotic proteins, in particular other eukaryotic proteins which are not ribosome-associated proteins, and ribosomes included in these extracts do not constitute at least 70-95% pure ribosomes (optionally in association with RNAs and/or ribosome-associated proteins) by weight, preferably at least 75% pure ribosomes (optionally in association with RNAs and/or ribosome-associated proteins) by weight, still preferably at least 80%, 85% or 90% pure ribosomes (optionally in association with RNAs and/or ribosome-associated proteins) by weight, most preferably 98%, 99% or 100% pure ribosomes (optionally in association with RNAs and/or ribosome-associated proteins) by weight.

Methods for purifying/isolating ribosomes are known by those skilled in the art. Common methods for isolating ribosomes are mainly based on differential centrifugation of cell lysate for separating the different cellular elements based on size and density. However, it is to be noted that it is highly preferred that the lysis buffer, as well as the sucrose cushion used to carry out differential centrifugation, comprise between 25 mM and 50 nM of KCl. Indeed, with a KCl concentration greater than 75 mM, the translational efficiency decreases dramatically. Therefore, preferably, the methods commonly used for isolating ribosomes are modified so that the lysis buffer and the sucrose cushion used to carry out differential centrifugation comprise no more than 25 mM to 50 nM of KCl. An example of method for isolating ribosomes are disclosed in Example 1 of the description described a typical method for purifying ribosomes. Briefly, a pellet of eukaryotic cells is produced by centrifugation, then it is diluted in a hypotonic buffer (buffer comprising Hepes 10 mM, CH₃CO₂K 10 mM, (CH₃CO₂)₂Mg 1 mM, DTT 1 mM), homogenized and centrifuged (e.g. at 16 000 g for 10 min) to yield the S10 supernatant extract which contains the ribosomes. S10 supernatant is centrifuged through a sucrose cushion (e.g. 1M sucrose in lysis buffer) for example 2 h 15 at 240 000 g. Then, sucrose solution is removed and the resulting pellet is resuspended in a suspension buffer (buffer comprising Hepes 20 mM, NaCl 10 mM, KCl 25 mM, MgCl₂ 1.1 mM, β-mercaptoethanol 7 mM).

In a preferred embodiment of the different aspects of the invention, the ribosomes isolated from eukaryotic cells are obtained from eukaryotic cells previously transfected, with a DNA (the DNA may be, for instance, a cDNA or a gene) coding for a protein of interest. Since the transcription occurs in cellulo and the translation begins within the cells, the mRNAs are in contact with the ribosomes. Thus, the ribosomes are isolated together with the mRNAs.

Furthermore, in the different embodiments of the different aspects of the invention, the eukaryotic cells from which the ribosomes are isolated can be eukaryotic cells previously transfected with i) a miRNA or a siRNA that targets a RNA of interest (e.g. a mRNA or a pre-mRNA), or ii) a DNA expressing said non coding RNA, so that the RNA of interest is not expressed (for instance, when the RNA of interest is a mRNA, protein translation does not occur). Thus, the cell-free translation system of the invention can be used for in vitro translation assay using ribosomes isolated from eukaryotic cells in which at least one endogenous protein, in particular a protein associated with the ribosomal fraction which is involved in the translational control which, has been depleted by RNA interference.

In the context of the invention, the term “red blood cells” (hereinafter abbreviated as “RBCs”) refers to all types of erythroid cells, in particular to mature erythrocytes or reticulocytes (immature red blood cells). Preferably, the red blood cells are reticulocytes.

The red blood cells may be obtained from any mammal, for instance from rabbit, human, rodent, horse, cow, dog, cat, goat, sheep etc. . . . . Preferably, the red blood cells are of human or rabbit origin. More preferably, the red blood cells are reticulocytes of rabbit.

The expression “a ribosome-depleted red blood cell lysate” refers to a red blood cells lystate treated, for example by ultracentrifugation, so that the ribosomes are pelleted and the supernatant of the lysate is free of ribosomes.

The centrifugation conditions, e.g. the centrifugal force necessary to pellet the ribosome are well known to the person skilled in the art.

By way of non non-limiting example, the ribosome-depleted red blood cell lysate may be obtained as follows: after lysis of the red blood cells, the lysate is centrifuged at 240 000 g for 2 hours and the lysate supernatant collected.

Methods for obtaining a red blood cell lysate are described for instance by Hunt and Jackson (Hamatol Bluttransfus, 14: 300-307, 1974) and Pelham and Jackson (Eur J Biochem., 67(1): 247-256, 1976).

Alternatively, when the ribosome-depleted red blood cell lysate is a ribosome-depleted rabbit reticulocyte lysate, the lysate of reticulocytes used to obtain the ribosome-depleted rabbit reticulocyte lysate may be the rabbit reticulocyte lysate marketed by Life Technologies (Ambion®) or PROMEGA®.

The red blood cell lysate may be treated with a nuclease (e.g. a calcium activated S7 microccocale nuclease) to dispose of the endogenous mRNAs that are contained into the reticulocytes.

In a preferred embodiment of the different aspects, embodiments and implementations of the invention, the red blood cell lysate used to obtain the ribosome-depleted red blood cell lysate is a rabbit reticulocyte lysate not treated with a nuclease ((e.g. a calcium activated S7 microccocale nuclease) (hereinafter abbreviated as “URRL”, stands for “untreated rabbit reticulocyte lysate”).

In the context of the invention, the expression “introducing a DNA into eukaryotic cells” refers to the introduction of a DNA (the sequence of a gene or the corresponding cDNA) into the cells so that under suitable conditions the DNA is expressed.

To introduce the DNA into the cells, the cells may be transformed, transfected, transduced or infectected with a plasmid, a vector, especially a vector of expression, or transduced with a virus vector, preferably a retrovirus vector, advantageously a lentivirus vector, comprising the DNA of interest, an AAV (“Adeno associated virus”), or inducible expression systems (systeme inductible TET OF/ON).

Conventional molecular biology, microbiology, and recombinant DNA techniques to product a DNA sequence, to clone a DNA sequence into a vector or a plasmid, and to introduce a DNA sequence into a cell are well known to the person having ordinarily skills in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription and Translation [B. D. Hames & S. J. Higgins, eds. (1984)]; Animal Cell Culture [R. I. Freshney, ed. (1986)]; Immobilized Cells and Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

The expression “a transcription reaction mixture” refers to a composition which comprises all necessary components required for achieving in vitro transcription of a DNA into the corresponding RNA.

In vitro transcription requires a DNA template containing a promoter (e.g. a prokaryotic phage promoter, for instance T7, T3 or SP6 promoter, or an eukaryotic virus promoter, for example CMV, SV40 promoter), ribonucleotide triphosphates rCTP, rATP, rUTP, rGTP, a buffer system, and an appropriate RNA polymerase. The components necessary to carry out an in vitro transcription are well known to the person skilled in the art, and are described in Ricci et al. (Nucleic Acids Res., 39(12): 5215-5231, 2011).

All references cited herein, including journal articles or abstracts, published patent applications, issued patents or any other references, are entirely incorporated by reference herein, including all data, tables, figures and text presented in the cited references.

The invention will be further illustrated by the following figure and examples. However, these examples and figure should not be interpreted in any way as limiting the scope of the present invention.

### BRIEF DESCRIPTION OF THE FIGURES

FIG. 1 illustrates the translational efficiency of different in vitro system.

Capped and polyadenylated in vitro transcribed RNA with the 5′UTR of β-globin (A) and (C) or GAPDH (B) upstream of the renilla luciferase was added to the following in vitro systems: Untreated Rabbit Reticulocyte Lysate (URRL), nuclease-treated Rabbit Reticulocyte Lysate (RRL), Wheat Germ Lysate (WG) and Human cell free system (HL) at the concentrations indicated on each panel. Translation was carried out for 30 minutes at 30° C. before determination of renilla activity as described in Example 1. Results are presented as mean+/−SD of three independent experiments.

FIG. 2 is a schematic representation of the experimental procedure used to fractionate the reticulocyte lysate. Briefly, it consists of isolating the ribosome fraction from the postribosomal supernatant by ultracentrifugation of RRL or URRL as depicted. The ribosomal fraction Rurrl (if obtained from URRL) or Rrrl (if obtained from RRL) is resuspended in buffer as described in Materials and Methods and the reconstituted lysate is assembled by mixing Su (if obtained from URRL) or Sr (if obtained from RRL) with the resuspended ribosomal fraction.

FIG. 3 illustrates the translational efficiency of the in vitro translation system of the invention.

Following the experimental procedure described above, homogeneous (Su+Rurrl; and Sr+Rrrl) or heterogeneous (Su+Rrrl; Sr+Rurrl) combinations were assembled and used for translation of the β-globin-renilla mRNA together with parental lysates (RRL and URRL). Su and Sr represent the reticulocyte supernatants after centrifugation with no added ribosomes. Values of the luciferase activity are given in arbitrary units and presented as mean+/−SD of three independent experiments.

FIG. 4 illustrates the translational efficiency of the in vitro translation system of the invention as a function of the ribosome concentration.

This figure represents the translational efficiency obtained from the β-globin-renilla construct translated in a reconstituted URRL (Su+Rrrl) that has been assembled with various concentrations of resuspended ribosomes: from 0.01× to 0.5×.

FIG. 5 is a schematic representation of the elaboration of the hybrid reconstituted lysate. The same experimental procedure as described above is applied to a cell extract to isolate the ribosomal fraction. A hybrid reconstituted system is then assembled with the supernatant from the URRL (Su) mixed with resuspended ribosomes from cells.

FIG. 6 illustrates the translational efficiency of the in vitro translation system of the invention using ribosomes isolated from different cells.

(A) The post-ribosomal supernatant from the untreated RRL (Su) was used in combination with ribosomes isolated from HeLa cells (Rh), Jurkat (Rj), RRL (Rrrl), untreated RRL (Rurrl) or wheat germ (Rwg) as indicated on the figure. The β-globin-renilla mRNA was then translated for 30 minutes in all combinations described above and in the untreated RRL as control. Values of the luciferase activity are given in arbitrary units and presented as mean+/−SD of three independent experiments.

(B) As above, ribosomes isolated from mouse stem cells (Rsc) were also used with the supernatant from the untreated RRL. Translation of the β-globin-renilla mRNA was carried out for 30 minutes at 30° C. before determination of renilla activity as described in Materials and Methods. Results are presented as mean+/−SD of three independent experiments.

FIG. 7 illustrates translation efficiency of different reporter mRNAs with the in vitro translation system of the invention.

Different reporter mRNA constructs including firefly, renilla luciferase and GFP coding region were translated in the hybrid system in the presence of labeled [³⁵S]-methionine. Protein products were resolved on a 12% SDS-PAGE and subjected to autoradiography. The position of the neo-synthesized polypeptides and the MW markers are indicated.

FIG. 8 illustrates the comparison of the in vitro translation efficiency of Pierce® with that of the in vitro translation system according to the invention.

The globin renilla reporter construct was used at different concentrations to program the HeLa cell lysate-based protein expression systems marketed by the company Pierce® (Human In Vitro Protein Expression Kit—RNA (Pierce® reference number 88857)) has been discontinued or the hybrid system as indicated on the figure. At the end of a 30 minutes incubation, expression of luciferase was determined and presented as mean+/−SD of two independent experiments realized in duplicates. The bottom panel summarizes the results and is plotted in a log. scale.

FIG. 9 is a schematic representation of the RNAs used in Example 4, the presence of the cap and poly(A) being indicated as +/+.

FIG. 10 illustrates the effects of capping and polyadenylation of the reporter mRNA on the efficiency of translation.

In vitro transcribed RNAs depicted above were used to program the RRL and the hybrid system composed of the URRL supernatant with ribosomes isolated from HeLa (Su+Rh) as indicated on the figure. Translation of the globin-renilla mRNA was carried out for 30 minutes at 30° C. before determination of renilla activity as described in Materials and Methods. Results are expressed as mean+/−SD of three independent experiments.

For clarity, a blow up picture of the values obtained with the glo−/− has been inserted below the graph.

FIG. 11 illustrates the comparison of the level of translation of an IRES-containing RNA with different cell free synthesis systems (“CFPS”).

The dual renilla luciferase bicistronic construct in which the EMCV IRES was inserted in the intercistronic spacer was translated in the Su+Rh hybrid system, the RRL and the WG (wheat germ) lysates. Both firefly and luciferase activities were determined after 30 minutes of incubation and are presented in separate graphs. Data are presented as mean+/−SD of three independent experiments.

FIG. 12 is an autoradiogram showing the production of both firefly and renilla luciferase from translation of the construct depicted above. The resulting mRNAs were expressed in the RRL (1 and 2) or the hybrid system (3 and 4) in the presence of L protease as indicated on the figure. Positions of the [³⁵S]-methionine labeled reporter genes are indicated on the left handside of the figure.

FIG. 13 illustrates the comparison of the level of translation of i) an IRES-containing RNA (CrPV-renilla) and ii) a RNA containing the beta-globin 5′UTR with Hela ribosomes isolated at different KCl concentrations.

Luciferase production from RNA constructs containing the CrPV (left panel) or the globin (right panel) 5′UTRs was measured in the hybrid system that had been assembled with ribosomal pellets isolated under different KCl concentrations ranging from 25 mM to 500 mM as indicated on the figure.

Data are presented as mean+/−SD of three independent experiments.

FIG. 14 illustrates the study of the recapitulation of the PV (poliovirus) IRES cell tropism.

(A) In vitro transcribed RNAs with the β-globin or the poliovirus 5′UTRs upstream of the renilla luciferase were translated in the RRL or a hybrid system assembled with HeLa ribosomes (Su+Rh). The results are expressed as a % of control which is represented by the value obtained for the globin-renilla construct in both RRL and Su+Rh and set to 100%. (B) In vitro transcribed RNAs described above were electroporated in BHK cells for 1 h before determination of the luciferase activity. The result is expressed as a % of control (globin), which was set to 100%.

(C) In vitro transcribed RNAs described above were translated in the hybrid system assembled with BHK ribosomes (Su+Rbhk). The result is expressed as a % of control (globin), which was set to 100%.

Results are presented as mean+/−SD of three independent experiments.

FIG. 15 illustrates that the effects of cell differentiation can be mimicked in the hybrid system.

(A) In vitro transcribed capped and polyadenylated RNA with the β-globin, GAPDH or Utrophin 5′UTRs upstream of the renilla luciferase were transfected by electroporation in undifferentiated and differentiated C2C12 cells as indicated. 1 h after RNA transfection, cells were lysed and translation of the globin-renilla (Glo), GAPDH-renilla (GAPDH) or Utrophin-renilla (Utro) mRNAs was determinated by measure of the renilla activity as described in Materials and Methods. Results are presented as mean+/−SD of three independent experiments. For clarity, a blow up picture of the values obtained with the Utro construct has been inserted below the graph.

(B) The hybrid system was assembled with supernatant from URRL (Su) with ribosomal fractions obtained from undifferentiated or differentiated C2C12 cells and used for translation of the Glo, GAPDH or Utro mRNAs as indicated. Data are presented as mean+/−SD of three independent experiments.

FIG. 16 illustrates the efficient depletion of an endogenous protein.

(A) Western blot analysis of a S10, S100 and ribosomal (C100) fractions of HeLa cells that have been treated with ShRNAs directed against GFP (control) or the DDX3 protein. Antibody recognizing DDX3 has been used to probe the membrane.

(B) In vitro transcribed capped and polyadenylated RNA with the β-globin or HIV-1 5′UTRs upstream of the renilla luciferase were transfected by electroporation in control or ShDDX3 treated HeLa cells as indicated. 1 h after RNA transfection, cells were lysed and translation of the globin-renilla (Glo) or HIV-1-renilla (HIV) mRNAs was determinated by measure of the renilla activity as described in Materials and Methods. Results are presented as mean+/−SD of three independent experiments.

(C) The hybrid system was assembled with supernatant from URRL (Su) with ribosomes obtained from control or ShDDX3 cells and used for translation of the Glo and HIV-1 renilla-luciferase mRNAs as indicated. Data are presented as mean+/−SD of three independent experiments.

FIG. 17 illustrates the translation from RNAs produced in cellulo by cells transfected with the corresponding cDNA.

(A) Schematic representation of the pathway used for RNA synthesis.

(B) Quantification by quantitative RT-PCR of the cytoplasmic RNAs resulting from transfection of 500 ng, 6 μg or 12 μg of β-globin-renilla cDNA.

(C) The hybrid system was obtained by mixing the Su with ribosomes isolated from HeLa cells that had been prealably transfected with 500 ng, 6 μg or 12 μg of globin renilla cDNA (as indicated). Once assembled on ice, the mixture has been incubated for 30 mn before measuring the luciferase activity. Results are presented as mean+/−SD of three independent experiments.

FIG. 18 illustrates the level of translation achieved according to different systems used (in vitro with rabbit reticulocyte lysate, ex vivo, with the “hybrid system” of the invention).

(A) Capped and polyadenylated in vitro transcribed RNAs with the β-globin or the c-myc 5′UTRs upstream of the renilla luciferase were added to the RRL. Translation was carried out for 30 minutes at 30° C. before determination of renilla activity as described in Materials and Methods.

(B) cDNA coding for β-globin or the c-myc 5′UTRs upstream of the renilla luciferase was transfected in HeLa cells. Cells were lysed 36 hours post-transfection and renilla activity was determined.

(C) Hybrid system constituted from Su mixed with ribosomes isolated from HeLa cells that had been prealably transfected with cDNA coding for the renilla luciferase driven by the globin or c-myc 5′UTRs (as indicated). Once assembled on ice, the mixture was incubated for 30 min before measuring luciferase activity.

Results are presented as mean+/−SD of three independent experiments.

FIG. 19 illustrates the level of translation achieved using ribosomes obtained from different mice organs and tissues (brain, lung, liver and heart) in comparison with the level of translation achieved using ribosomes obtained from HeLa cells, Jurkat cells or stem cells.

FIG. 20 illustrates the detection, by electrophoresis, of viral proteins translated from mRNAs and ribosomes obtained from A549 cells infected with influenza virus (R_{A549-PR8}) or non-infected A549 cells (R_{A549}) using the translation system of the invention.

### EXAMPLE 1

### Materials and Methods

DNA Constructs

The globin, GAPDH, PV, HIV1, c-myc 5′UTR, EMCV, CrPV and Utrophin 5′UTRs were obtained by PCR using the p0-glo-renilla, p0-GAPDH-renilla p0-EMCV-renilla, p0-PV-renilla, p0-HIV1-renilla, p0-CrPV-renilla (Soto Rifo et al., Nucleic Acids Res., 35(18): e121, 2007; Soto-Rifo et al., Nucleic Acids Res., 2011; Soto-Rifo et al., Embo. J., 31(18): 3745-3756, 2012), c-myc pRMF (Evans et al., Oncogene, 22(39): 8012-8020, 2003), pGL4.14CMV 5′UTR mUTROPHIN (Miura et al., J. Biol. Chem., 280(38): 32997-33005, 2005) respectively using specific primers containing PvuII restriction site and T7 promoter and HpaI restriction site (for sense primers) and BamHI restriction site (for antisense primers). PCR products were digested and cloned in p1-renilla and pCDNA3.1-renilla backbone vectors previously digested by PvuII and BamHI or HpaI and BamHI restriction enzymes respectively. The pCDNA3.1 vector was modified after the CMV promotor to minimize the number of nucleotides added upstream of the 5′UTR. Position of the +1 transcription site was controlled by rapid amplification of cDNA extremity (RACE) (Ambion kit). P1-bicistronic construction was cloned with the combination of the simple digest p0-β-globin-firefly vector (AflII restriction site) and EMCV-renilla insert obtained by PCR using the p1-EMCV-renilla.

pRS-shCtrl and pRS-shDDX3 vectors were respectively generated by a 5′-GCAGCACGACTTCTTCAAGTTCAAGAGACTTGAAGAAGTCGTGCTGC-3′ (SEQ ID NO: 1) and 5′-GATGCTGGCTCGTGATTTCTTTCAAGAGAAGAAATCACGAGCCAGCATC-3′ (SEQ ID NO: 2) (target sequence in bold) insertion between the BglII/HindIII sites of the pRetroSuper vector (OligoEngine) following supplier's instructions.

In Vitro Transcription

RNAs were transcribed using the T7 RNA polymerase from templates linearized either at the AflII for Polyadenylated RNAs or at the EcoRV sites for non-polyadenylated RNAs. Uncapped RNAs were obtained by using 1 μg of linear DNA template, 20 U of T7 RNA polymerase (Promega), 40 U of RNAsin (promega), 1.6 mM of each ribonucleotide triphosphate, 3 mM DTT in transcription buffer (40 mM Tris-HCl (pH 7.9), 6 mM MgCl₂, 2 mM spermidine and 10 mM NaCl). For capped mRNAs, the rGTP concentration was reduced to 0.32 mM and 1.28 mM of m⁷GpppG cap analogue (New England Biolabs) was added. The transcription reaction was carried out at 37° C. for 2 h and the mRNAs were precipitated with ammonium acetate at 2.5M final concentration. The RNA pellet was then resuspended in 30 μL RNAse free water and RNA concentration was determined by absorbance using Nanodrop technology. RNAs integrity was checked by electrophoresis on non-denaturing agarose gel.

Cell Culture and Nucleic Acid Transfections

Hela, C2C12, BHK and Jurkat cells were obtained originally from American Tissue Type Culture Collection. Mouse stem cells were kindly donated by D. Aubert (IGF-Lyon, France).

Hela, BHK and C2C12 cells were typically grown in DMEM containing 10% fetal calf serum (FSC) supplemented with 50 U/ml of penicillin, 50 μg/ml of streptomycin (PS) under a humidified atmosphere containing 5% CO2 at 37° C. C2C12 differentiation is induced by DMEM containing 2% horse serum. Jurkat cells were grown in (RPMI) containing 10% FSC supplemented with 50 U/ml of penicillin, 50 μg/ml of streptomycin, 10 mM Hepes (pH 7.2-7.5), 2 mM L-Glutamine and 1 mM pyruvate under a humidified atmosphere containing 5% CO2 at 37° C. Mouse stem cells were grown in GMEM containing 10% FSC supplemented with 50 U/ml of penicillin, 50 μg/ml of streptomycin, 1% MEM unessential amino acids (Gibco), 1 mM sodium pyruvate, 2 mM L-Glutamine, 40 μM β-mercaptoethanol and 400 μL Leukemia inhibitory factor (LIF) (Chemicon) under a humidified atmosphere containing 7.5% CO2 at 37° C. For DDX3 knock-down, stable clone of Hela cells were obtained by transfecting the pRS-shCtrl or pRS-shDDX3 vectors that were maintained and selected in DMEM growth media supplemented with 1 μg/mL puromycin, 10% FSC and 1% PS.

DNA Transfection

Hela cells were transfected with 12 μg of total DNA containing 500 ng, 6 μg or 12 μg of interest plasmid DNA per 175 cm² containing 1.5·10⁷ cells using cationic polymers (JetPEI from Polyplus) as specified by the manufacturer. Cells were lysed 24 h to 48 h after transfection either to determine luciferase activity or to pellet and isolate the ribosomes as indicated in the manuscript (see below).

RNA Transfection

Hela, C2C12 and BHK cells were electroporated with 100 ng of in vitro synthesized mRNAs (see below) for 10⁵ cells with the Neon™ system (life technology) following supplier's indications. Cells were lysed 1 h after transfection and luciferase activity was determined.

Ribosome Purification

All following steps were performed at 4° C.

S10 Preparation:

The pellet of 10⁸ cells was diluted in an isovolume of lysis buffer (buffer R: Hepes 10 mMM, CH₃CO₂K 10 mM, (CH₃CO₂)₂Mg 1 mM, DTT 1 mM). The cell suspension was homogenized by potter and centrifuged at 16 000 g for 10 mn to yield the S10 supernatant extract.

Ribosomal Fraction:

300 μl of S10 preparation was centrifuged through a sucrose cushion (1M sucrose in buffer R) for 2 h 15 at 240 000 g. After removal of the sucrose solution, the resulting pellet is gently rinsed in buffer R2 and resuspended in buffer R2 (buffer R2: Hepes 20 mM, NaCl 10 mM, KCl 25 mM, MgCl₂ 1.1 mM, β-mercaptoethanol 7 mM) and stored at −80° C.

Fractionation of the Reticulocyte Lysate:

After centrifugation of 1 ml of URRURRL, 950 μl of post-ribosomal supernatant is collected, frozen and stored at −80° C. The ribosomal pellet is rinsed in buffer R2 and resuspended in buffer R2 as above.

Preparation of URRL and In Vitro Translation Assays

The method used was identical to that described (Soto Rifo et al., Nucleic Acids Res., 35(18): e121, 2007) and can be briefly summarized as follow: 1 ml of untreated RRL (URRL) was supplemented with 25 μM Hemin (Fluka), 25 μg creatine phosphokinase (Sigma Aldrich), 5 mg creatine phosphate (Fluka), 50 μg of bovine liver tRNAs (Sigma Aldrich) and 3 mM of D-glucose (Sigma Aldrich).

For reconstitution of the hybrid system, 5 μl of S100 supernatant isolated from RRL or URRL as indicated on the figure was mixed with 1 μg of C100 derived from cultured cells (HeLa, Jurkat, BHK, C2C12, mouse stem cells) or reticulocyte lysate (RRL or URRL).

In vitro transcribed RNAs were translated at 2.7 nM unless specified in the figure legend in a final volume of 10 μl of lysate (either crude or reconstituted as indicated on figures) supplemented with 75 mM KCl, 0.75 mM MgCl2, 20 μM amino acids mix. The translation reaction is left incubated for 30 mn at 30° C. before the reaction is stopped by addition of renilla lysis buffer (Promega). When adequate, [³⁵S]-methionine labeled radioactive proteins were translated in presence of 20 μM of amino acids mix minus methionine and 5 μCi of [³⁵S]-methionine (Perkin Elmer) for 30 mn before the reaction was stopped by addition of SDS loading buffer.

Preparation of L-Protease

The L-protease from the foot and mouth disease virus (FMDV) or GFP for control was produced by in vitro translation using RRL as previously described (Ohlmann et al., RNA, 5(6): 764-778, 1999) and 0.1 μl (per 10 μl final volume of lysate) was added and incubated for 10 mn at 30° C. prior to the start of the translation reaction.

Western Blot

Samples were resolved on a 10% or 12% SDS-PAGE, transferred to PVDF membrane and blotted using anti DDX3 (Abcam), eIF4G (kindly provided by Dr. Simon Morley, University of Sussex, United Kingdom).

Renilla Activity

Renilla activity was measured using the renilla luciferase Assay System (Promega Co, Madison, Wis., USA) in a Mithras (Berthold technologies) with 50 μl substrate injection and 10 seconds of signal integration program.

RNA Extraction and RT-qPCR

Cytoplasmic RNA extraction and RT-qPCR was performed as previously described (Ricci et al., Nucleic Acids Res., 39(12): 5215-5231, 2011).

### EXAMPLE 2

### Comparison of Translational Efficiency in Some CFPS

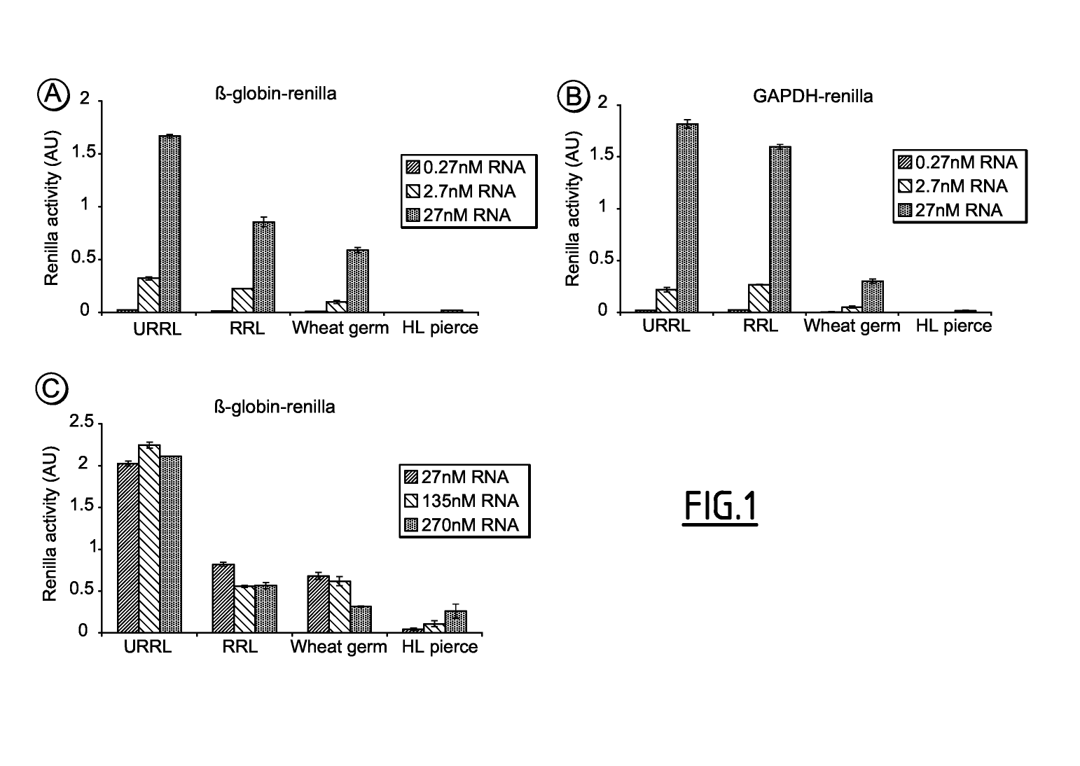

Given the diversity of in vitro translation systems, the inventors wanted to compare the most commonly used ones such as the rabbit reticulocyte (treated or not with the S7 nuclease), the wheat germ and the newly available Human lysates from Pierce which is prepared from HeLa cell extracts. For each of these lysates, their ability to translate in vitro transcribed mRNAs has been monitored. The renilla luciferase whose expression was driven either by the β-globin (50 nts) or the GAPDH (102 nts) 5′ untranslated region (5′UTR) was used. These RNA constructs harbor a m⁷GTP cap moiety together with a 50 adenylate residue poly(A) and were translated in either the crude rabbit reticulocyte lysate (URRL), the micrococcal treated rabbit reticulocyte lysate (RRL), the wheat germ lysate (WG) and the human in vitro protein expression system (HL pierce). Results obtained are summarized in FIG. 1A for the mRNA that is driven by the β-globin 5′UTR and 1B for the one driven by the GAPDH leader. For both mRNAs, the best translational efficiency was obtained in the rabbit reticulocyte derived systems, namely URRL and RRL. This was observed at all RNA concentrations tested (0.27; 2.7 and 27 nM) and even with saturating amounts of exogenous mRNAs added (FIG. 10). It is noteworthy that addition of high concentration of mRNAs (from 27 nM to 270 nM) was mostly beneficial to the HL systems in terms of global translational efficiency (see FIG. 10) but it remained below the level of activity observed in the RRL.

### EXAMPLE 3

### Design of a Novel Hybrid In Vitro Translation System

The aim of the invention was to design a novel in vitro cell free system that can combine translational efficiency and characteristic features of living cells. To do that, the creation of a hybrid translational system between components of the rabbit reticulocyte lysate with those derived from cultured cells has been considered.

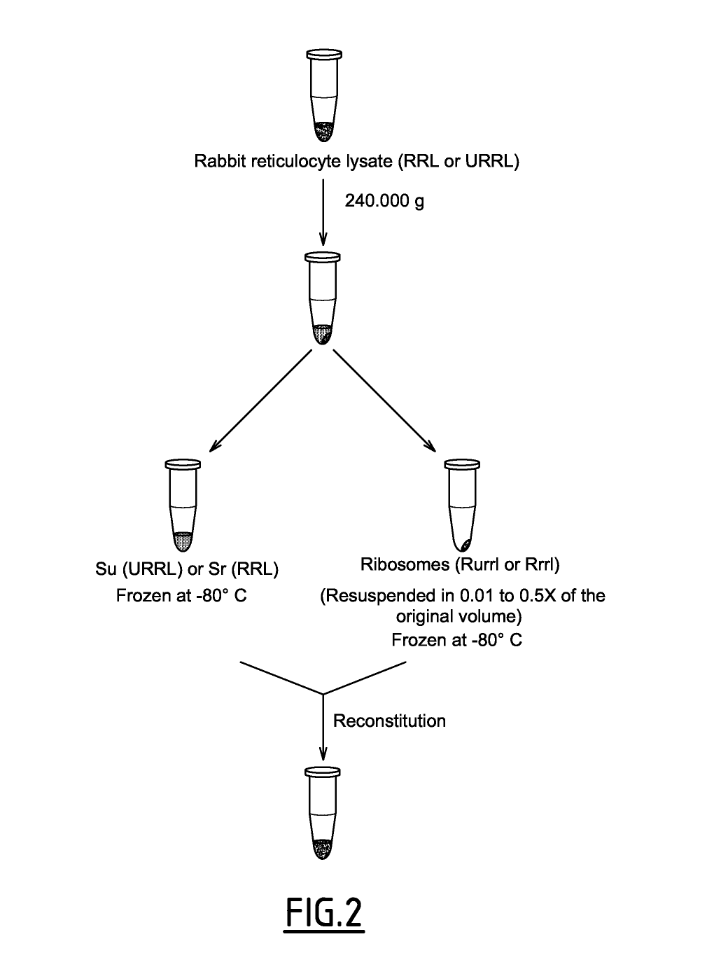

In order to do this, the inventors first adapted the method developed by Rau et al, in 1998 which consists of fractionating the rabbit reticulocyte lysate into a S100 supernatant and the ribosomal pellet (Rau et al., Methods Mol Biol 77: 211-226, 1998) by centrifuging at 240 000 g for 135 mn. It resulted in the separation of the cytosolic components of the protein synthesis apparatus from ribosomes associated one: both fractions could be rapidly frozen and stored at −80° C. for several months (see FIG. 2 and materials and methods).

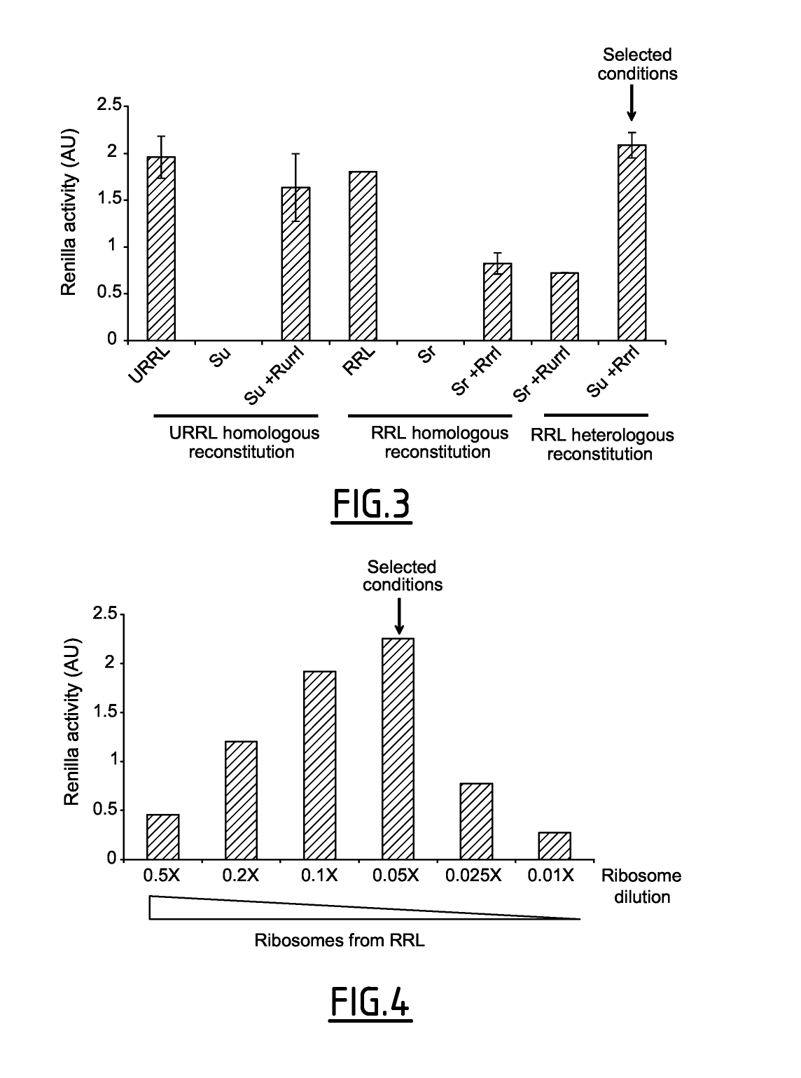

In a first attempt, the inventors mixed components from the URRL with those of the RRL in the 4 possible combinations (Su+Rurrl; Sr+Rrrl, Sr+Rurrl and Su+Rrrl) that were all used to translate the Renilla construct driven by the β-globin 5′UTR (FIG. 3). It could first be observed that the supernatant fraction (without ribosomes) did not yield any luciferase activity (see Su and Sr) confirming that the bulk of ribosomes were removed during the centrifugation step.

The renilla construct was translated in both parental lysates (URRL and RRL) and efficiency was compared with the homologous reconstituted systems (Su+Rurrl and Sr+Rrrl). Although translational activity was of similar magnitude for both URRL and RRL, it was not the case once the systems were reconstituted with the Su+Rurrl being about twice as efficient as the reconstituted Sr+Rrrl. Interestingly, the inventors also observed that the Su+Rrrl was the best combination for heterologous systems with a translational yield comparable to that obtained with the parental lysates (FIG. 3). These data suggest that the S100 supernatant from the untreated lysate (Su) is the most efficient fraction and will be selected to serve for the basis of the hybrid lysate (see later).

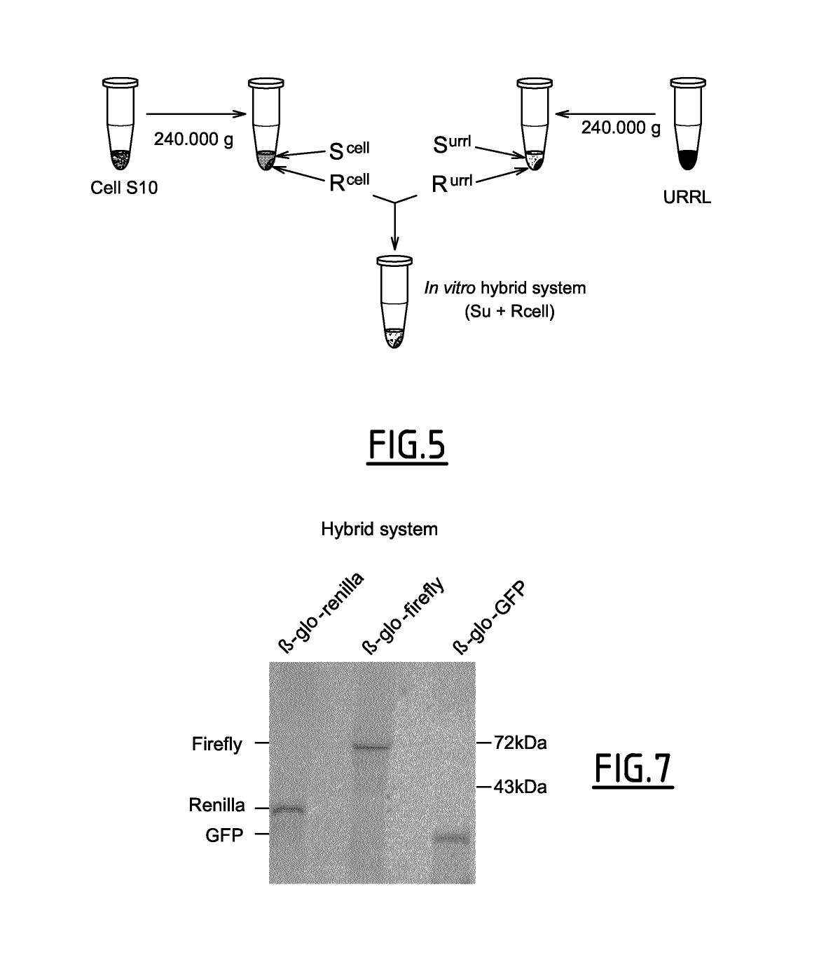

In order to optimize the system, the optimal amount of ribosomes (from RRL) that can be mixed to the untreated lysate supernatant (Su) was determined and it was found that 1 μg of pelleted ribosomes yielded the best translational efficiency (FIG. 4) which corresponds to only 0.05× ( 1/20) of the parental ribosome concentration.

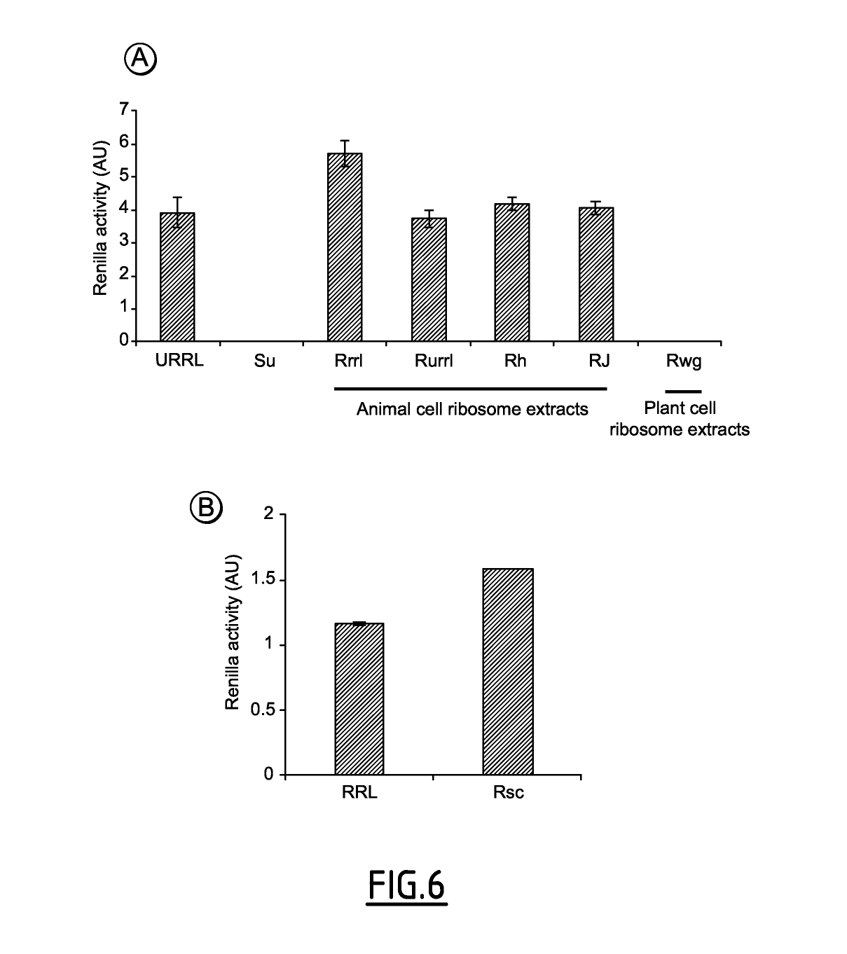

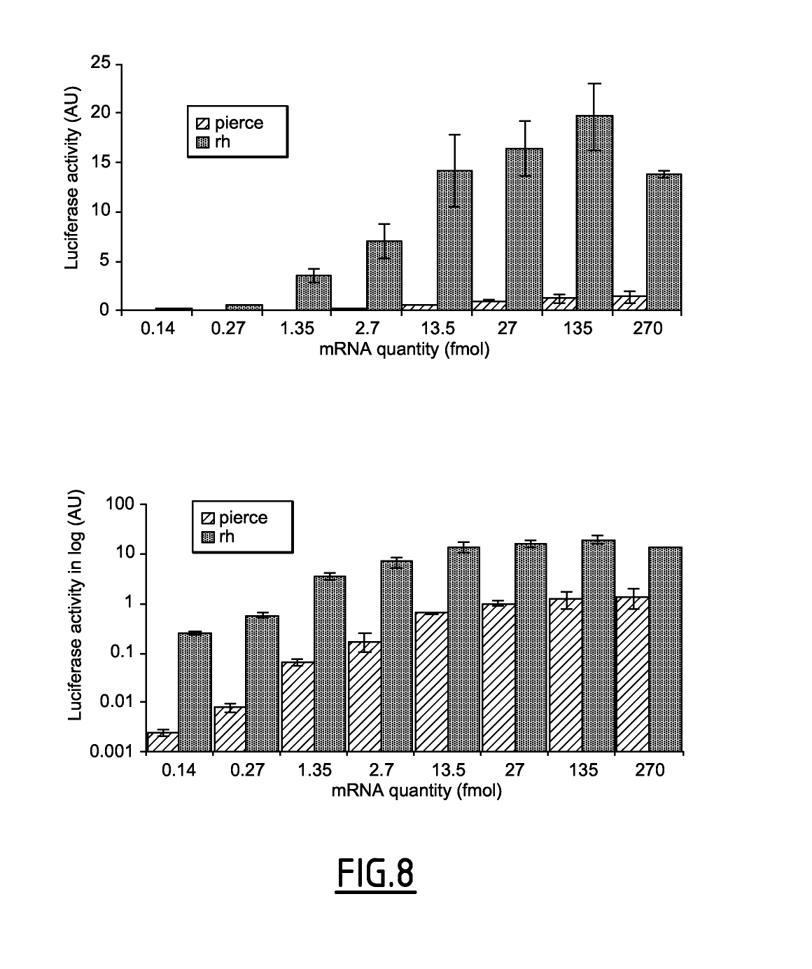

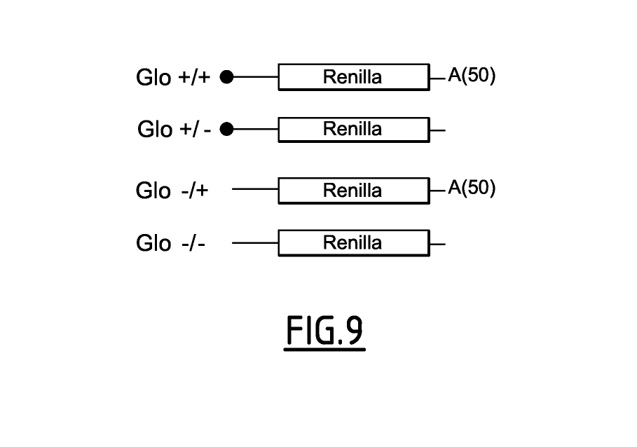

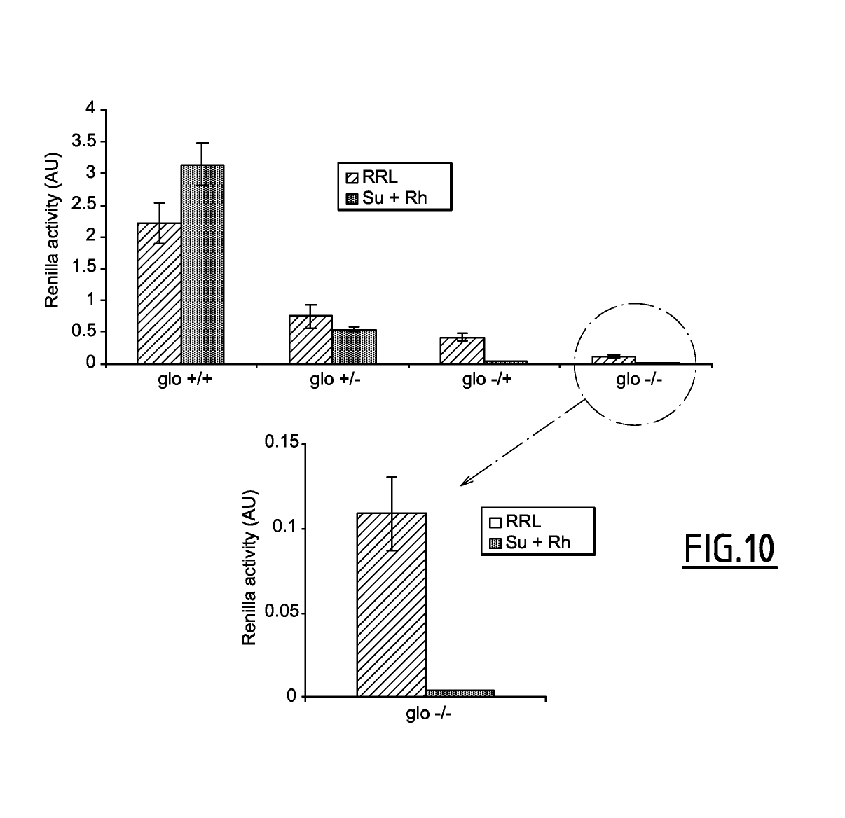

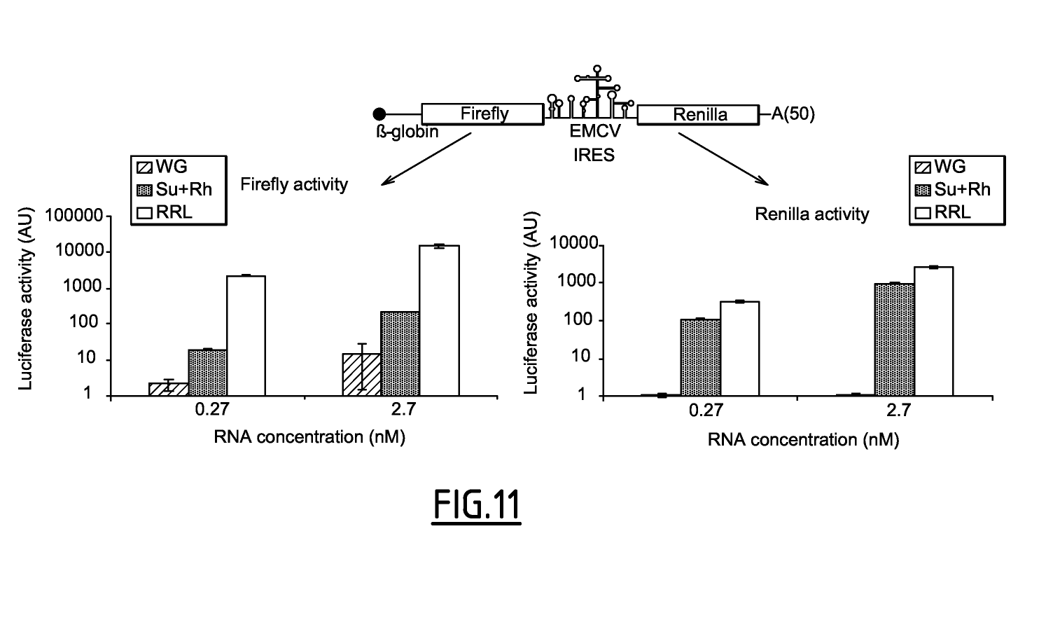

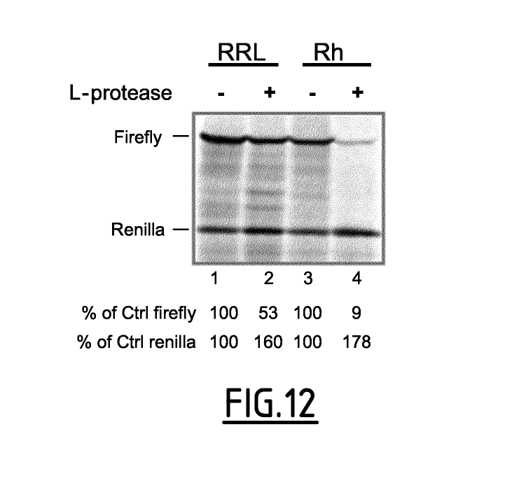

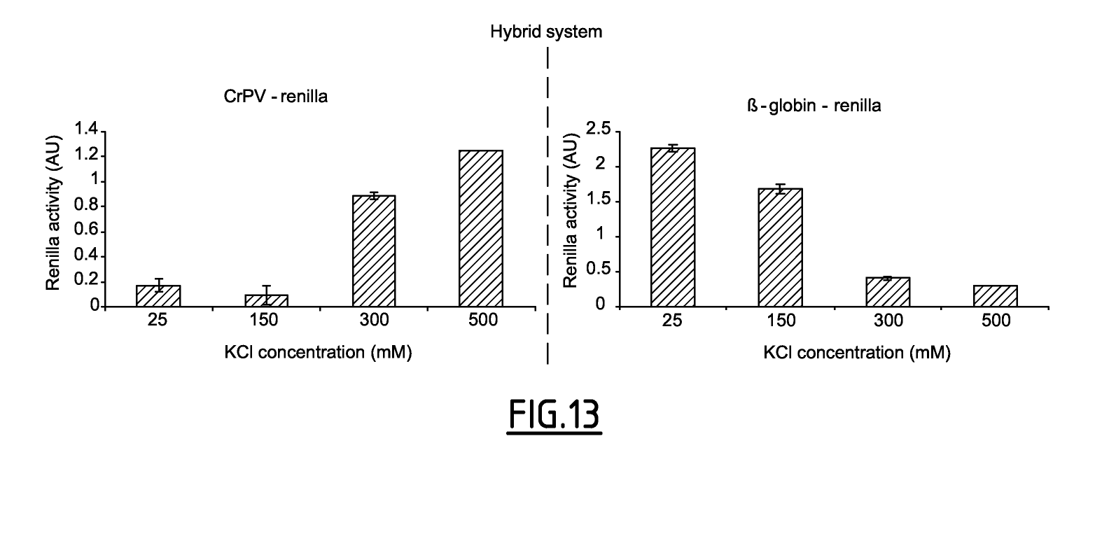

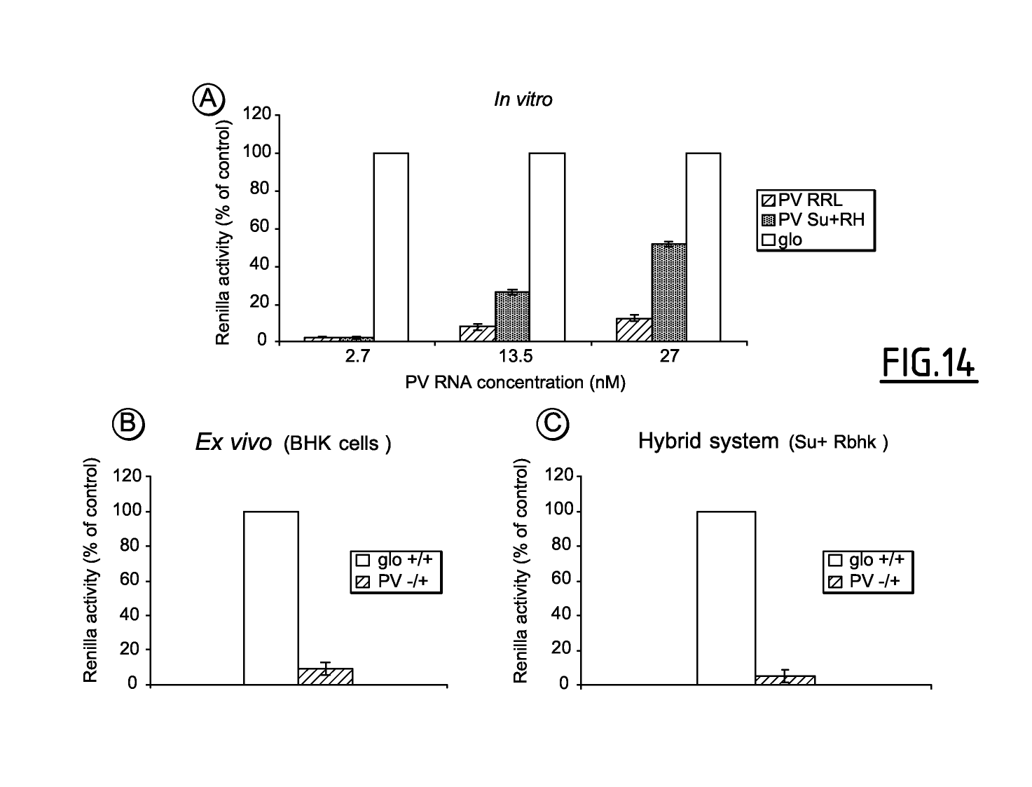

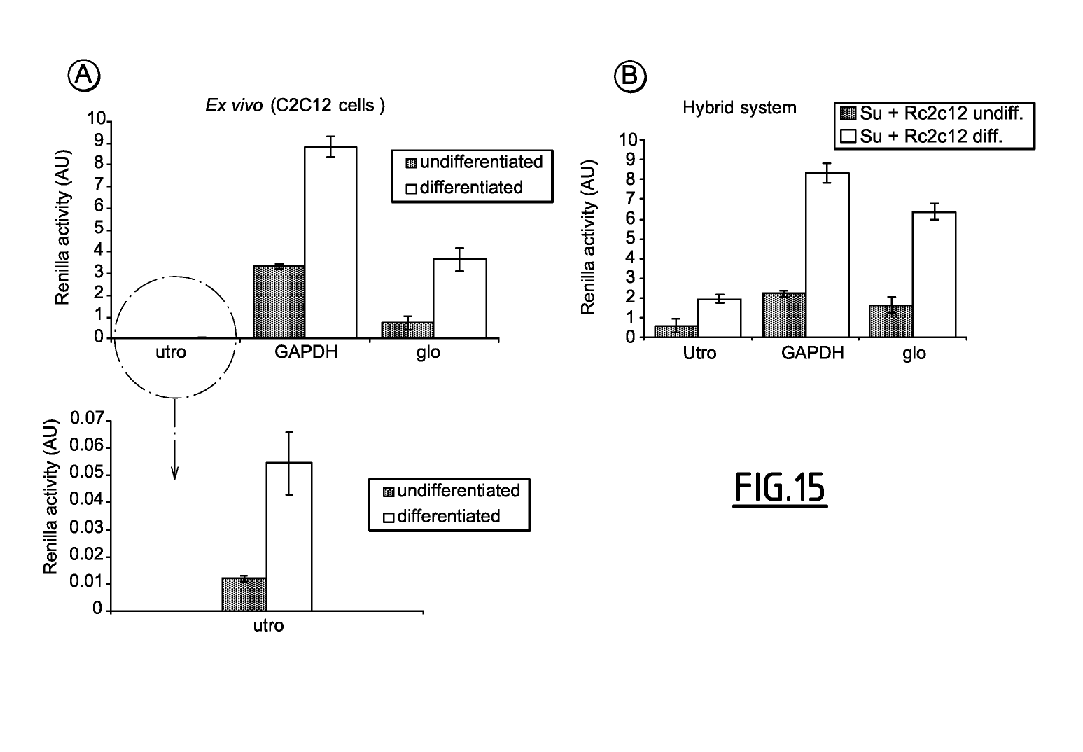

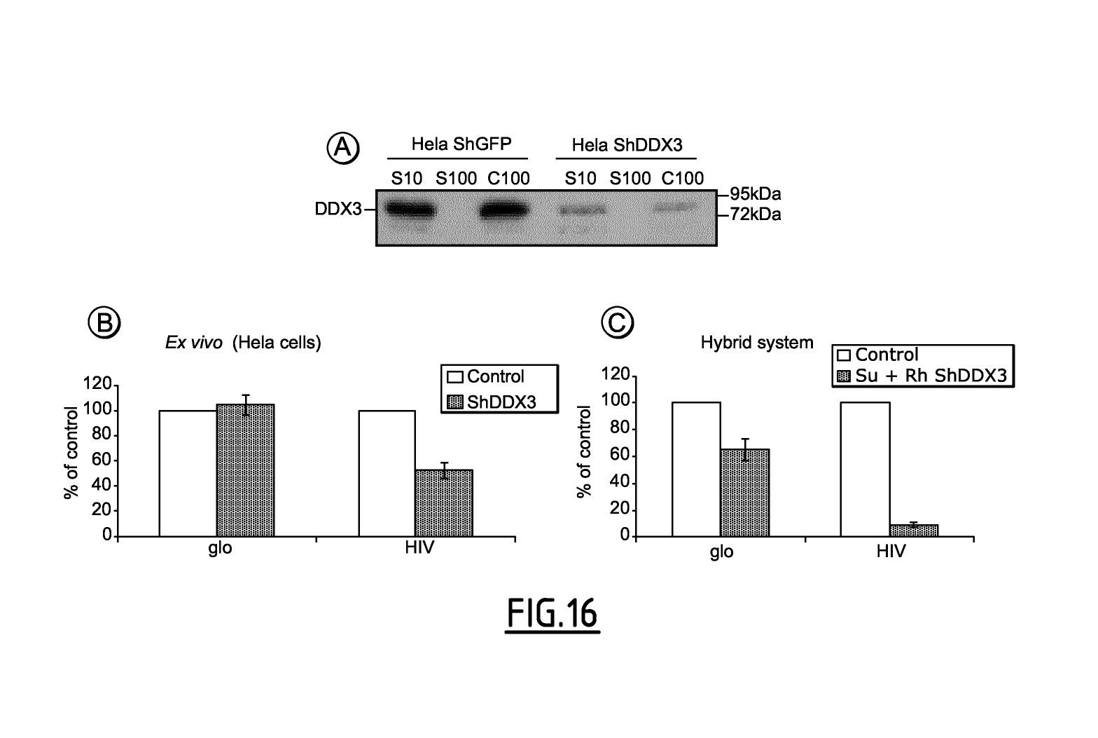

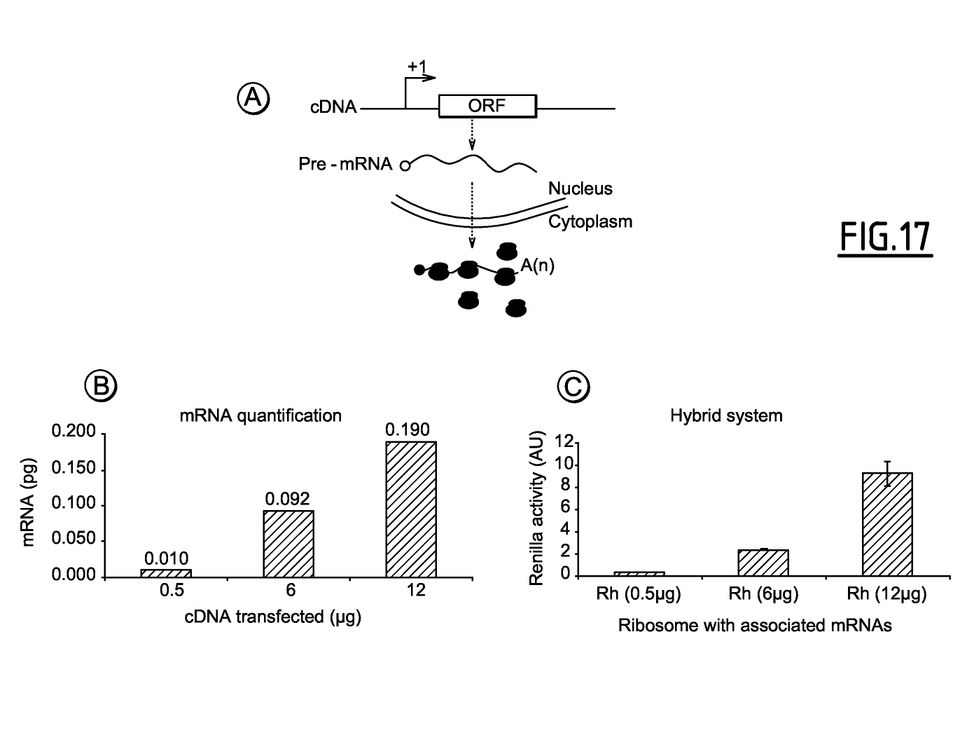

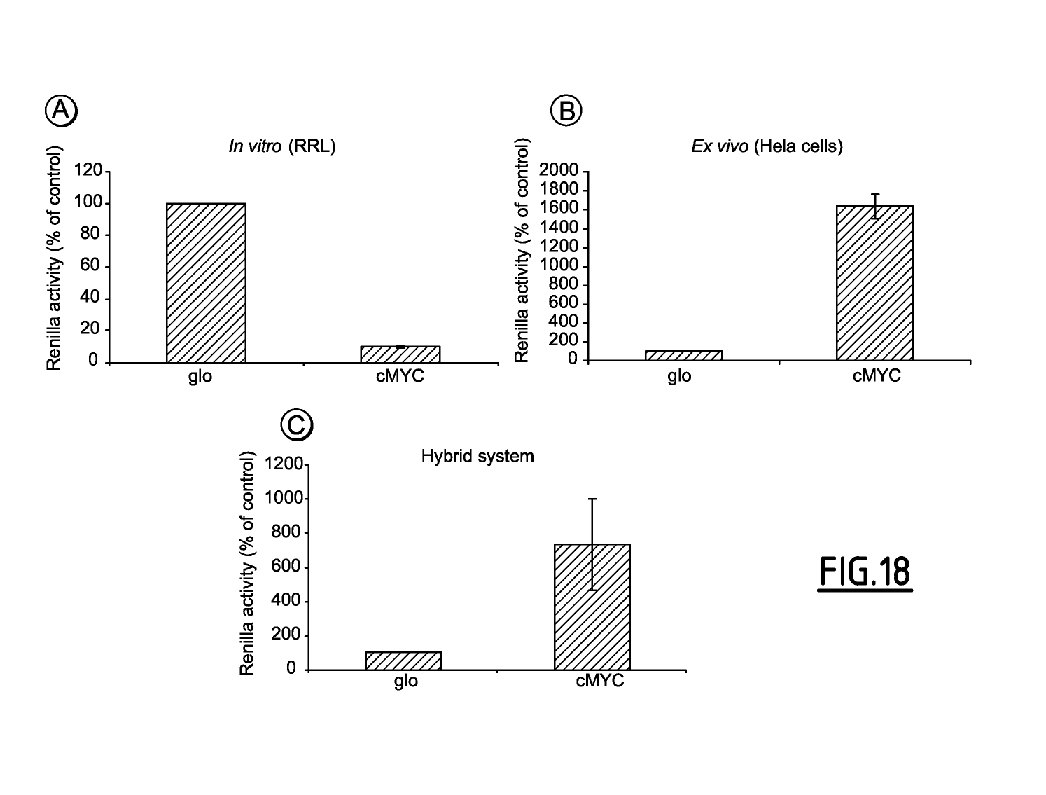

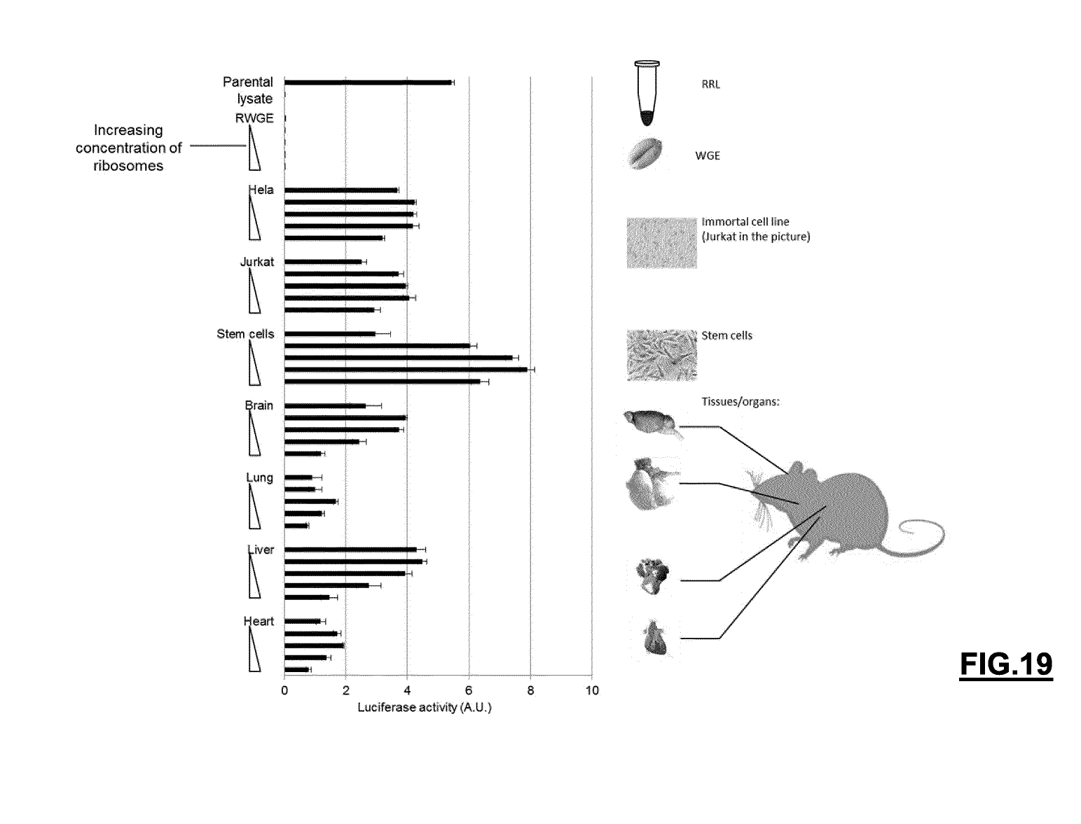

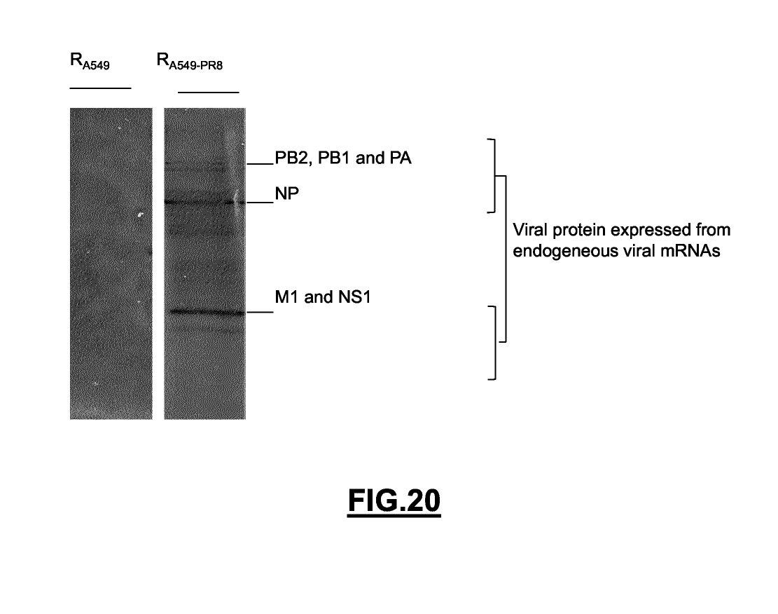

These data were used to elaborate a hybrid reconstituted cell free system in which the ribosomes are isolated from cultured cells whereas the S100 supernatant is provided by the URRL (see cartoon, FIG. 5). Two human cell lines (HeLa and Jurkat), the RRL, URRL and the wheat germ lysate were used to yield 5 ribosomal pellets (Rurrl for ribosomes of the URRL; Rrrl for ribosomes of the RRL; Rh for ribosomes obtained from the Hela lysate S10; Rj for ribosomes obtained from Jurkat cells and Rwg for ribosomes from the WG lysate). The post-ribosomal supernatant of the URRL was used to generate the cytosolic fraction (Su for supernatant of the URRL). Hybrid reconstituted systems were assembled by mixing the ribosome fractions (R) with the S100 supernatant from the URRL (Su) in all possible combinations. In these systems, 2.7 nM of the globin-renilla reporter gene was translated for 30 mn before analysis of renilla activity. Data showed that all combinations with mammalian ribosomes resulted in efficient protein synthesis to a level comparable to that observed with the reconstituted RRL or the parental lysate (FIG. 6A, compare Rrrl and URRL with Rurrl/Rh/Rj). However, virtually no renilla activity could be detected with wheat germ ribosomes (FIG. 6A, see Rwg). These experiments were extended to ribosomes isolated from ‘non specialized’ cells such as mouse stem cells that were obtained following the same experimental procedure (FIG. 6B). Upon reconstitution of hybrid system containing stem cell ribosomes, translation of the globin-renilla construct was just as efficient as in the RRL system (FIG. 6B, compare RRL and Rsc).

Furthermore, these experiments were extended to ribosomes isolated from mice organs and tissues (i.e. brain, heart, liver and lung) that were obtained following the same experimental procedure. It can be observed on FIG. 19 that protein synthesis in the hybrid system reconstituted with ribosomes from organs was of comparable efficiency to that with cell lines. Therefore, this indicates that ribosomes can be obtained from any vertebrate organs or tissues including pathogenic ones derived from tumours, metastasis or biopsy samples.

Because only minute amounts of renilla are needed for efficient detection and quantification, it could be argued that protein production in the hybrid system is not that efficient after all. Moreover, as discussed in the introduction, it could be of interest to use a CFPS in which protein production occurs in a sufficient yield to be able to detect the neo synthesized protein by [³⁵S]-methionine incorporation. Thus, the hybrid system (Su+Rh) with reporter mRNAs that are routinely used in laboratories (GFP, renilla and firefly luciferase genes driven by the β-globin 5′UTR) in the presence of radioactive [35S]-methionine was programmed. At the end of a 30 mn incubation, proteins were resolved on SDS-PAGE and the dried gel submitted to autoradiography (FIG. 7). This shows that the protein products from all reporter genes were detected as a single sharp band indicating that protein synthesis had occurred efficiently and faithfully with neither truncated aborted polypeptides nor shorter diffuse isoforms that could be indicative of premature protein degradation or ribosome drop-off.

Finally, the efficiency of the manufactured expression system based on HeLa lysates marketed by the company Pierce has been compared with the hybrid lysate containing HeLa ribosomes. For this, the globin renilla reporter gene was translated at a broad spectrum of RNA concentration and renilla activity was measured after 30 minutes. The results are presented in FIG. 8 and show that for all RNA concentrations tested, our system was several fold more efficient than the pierce lysate. The bottom panel summarizes these data that are plotted on a logarythmic scale.

Taken together, these data validate the use of ribosomes isolated from mammalian cell lines in the context of the rabbit reticulocyte lysate.

### EXAMPLE 4

### The Hybrid System Recapitulates Cap/Poly(A) Synergy and Support IRES-Driven Translation

One of the major drawback of the nuclease treated rabbit reticulocyte lysate is that it fails to recreate the selective advantage conferred by the addition of the poly(A) tail on transcripts and does not recapitulate the cap/poly(A) synergistic effect on translation (Borman et al., Nucleic Acids Res., 25: 925-932, 2000). Such a property can be found in the crude RRL but use of the latter is restricted by the synthesis of endogenous globin and lipoxygenase which can interfere with ectopic gene translation both at the level of RNA and protein production (Soto Rifo et al., Nucleic Acids Res., 35(18): e121, 2007).

Therefore, the next step was to investigate how the hybrid system could recapitulate the effects of capping and polyadenylation. For this, the inventors used in vitro transcribed globin renilla genes which were produced in the four possible combinations, capped/polyadenylated (+/+), capped/non polyadenylated (+/−), uncapped/polyadenylated (−/+) and uncapped/non polyadenylated (−/−) as indicated (see cartoon on FIG. 9). The resulting RNAs were added to the hybrid SuRh (Supernatant of URRL with ribosomes from HeLa) or the nuclease treated lysate (RRL) as control. As it could be seen on FIG. 10, the level of translation in both systems was pretty similar when the mRNA harbored a cap at its 5′ end. However, omission of this cap resulted in the virtual absence of activity in the hybrid system (see −/+) and this was even further evidenced when the poly(A) tail was missing (see −/−) whereas both combination were still translated in the RRL, albeit to a lower efficiency. This indicates that the hybrid system reconstitutes a more physiological cap/poly(A) driven translational environment.

Many studies have focused on internal initiation which is the mechanism used by some mRNAs to recruit ribosomes at an internal position with no need for a 5′ cap structure (Balvay et al., Biochim. Biophys. Acta, 2009). Some in vitro systems are very inefficient to support internal initiation and this was notably shown for the WG lysate in which picornaviral RNAs are poorly expressed (Woolaway et al., J. Virol., 75(21): 10244-10249, 2001).

To assess translation of IRES-containing RNAs, the inventors used a bicistronic construct coding for the firefly (first gene) and the renilla (second gene) in which the Encephalomyocarditis virus IRES (EMCV) was inserted in the intercistronic spacer. As seen on FIG. 11, production of the first gene firefly was efficient in all three CFPS tested (WG, RRL and Rh) whereas renilla synthesis which results from IRES expression, was only observed in the RRL and Rh lysates (right panel) but not the WG as previously shown (Woolaway et al, 2001).

Then, it was checked whether the hybrid system could recapitulate some selective selections that are encountered in the course of picornaviral replication. One of those is created by the cleavage of the initiation factor eIF4G by the virally encoded L protease from the picornavirus FMDV (Ohlmann et al., Nucleic Acids Res., 23(3): 334-340, 1995; Ziegler et al., J Virol 69(6): 3465-347, 1995b). In infected cells, such a proteolytic event results in the collapse of cellular cap-dependent protein synthesis whereas translation from the viral IRES is stimulated (Devaney et al., J. Virol., 62(11): 4407-4409, 1988; Hambidge et al., Proc. Natl. Acad. Sci. USA, 89(21): 10272-10276, 1992; Krausslich et al., J. Virol., 61(9): 2711-2718, 1987; Ziegler et al., J. Virol., 69(6): 3465-3474, 1995b). Interestingly, the addition of the L protease in the RRL results in a quite attenuated effect of the viral enzyme in comparison to what happens in living cells; as such, in the RRL, cap-dependent translation is decreased and a mild stimulation of the picornaviral IRES is generally observed (Ohlmann et al., Nucleic Acids Res., 23(3): 334-340, 1995; Soto Rifo et al., Nucleic Acids Res., 35(18): e121, 2007). Therefore, the in vitro translated L protease to both the RRL and the hybrid system prior to translation of the firefly-EMCV-renilla bicistronic construct used above was added. Western blot analysis showed that eIF4G was similarly proteolysed in both assays (data not shown) and protein production was determined by [³⁵S]-methionine incorporation in order to better compare the variations of expression of the two genes (FIG. 12). This shows that the addition of L protease stimulates renilla production whereas firefly synthesis was diminished in the RRL and virtually abolished in the hybrid system (FIG. 12, lanes 2 and lanes 4).

Finally, the ability of the hybrid system to reproduce very peculiar translational conditions that can be encountered with some dicistrovirus IRESes such as the intergenic IRES from the Cricket Paralysis Virus (CrPV) was evaluated. Initiation on this RNA takes place by direct binding of the 40 S ribosomal subunit in the absence of any initiation factors (Wilson et al., Cell, 102(4): 511-520, 2000). This confers to the virus a selective advantage over cellular translation during infection, as it was shown that drastic physiological conditions such as elF2 phosphorylation, stress or initiation factors depletion enhances CrPV translation (Garrey et al., J. Virol., 84(2): 1124-1138, 2010).

Therefore, the HeLa ribosomes at different KCl concentrations ranging from 25 mM to 500 mM were isolated. At the highest salt concentration, most of the ribosomes associated factors including elFs are washed out and not present anymore in the ribosomal pellet (data not shown). Hybrid systems were assembled with these ribosomes purified under increasing chaotropic conditions and used to measure translation driven by the CrPV IRES (FIG. 13, left panel). This clearly shows that expression of the latter was stimulated by the loss of ribosome associated factors (FIG. 13, left panel, see 300 and 500 mM KCl concentrations). This sharply contrasts with the β-globin-renilla mRNA which was severely inhibited under the same conditions (FIG. 13, right panel). These data confirm that translation of the CrPV can benefit from conditions where initiation factors are inactivated or present in severely limiting concentrations as previously described (Garrey et al., J. Virol., 84(2): 1124-1138, 2010; Pestova et al., Genes Dev., 17(2): 181-186, 2003); this further shows that the hybrid system can be used for translation of very specialized mRNAs under specific physiological conditions.

### EXAMPLE 5

### The Hybrid System Recapitulates Cellular Tropism

The present data indicate that the addition of ribosomes isolated from HeLa cells confer cap/poly(A) synergy to the lysate, restitute the ability to internal initiation and conserve the good translational efficiency of the reticulocyte lysate. Therefore, it was investigated whether the hybrid system could restitute cellular tropism. It has been shown that some IRES-containing mRNAs, notably those driven by the poliovirus IRES, were poorly expressed in the reticulocyte lysate and this can be partially rescued by the exogenous addition of HeLa S10 cell extracts to the reticulocyte lysate (Borman et al., Nucleic Acids Res., 25: 925-932, 1995). Thus, the expression of the PV IRES in the reticulocyte lysate and in the hybrid system which was assembled with ribosomes from HeLa cells (Su+Rh) was compared. As a control, the β-globin-renilla construct was also translated in the two assays (RRL and hybrid system). Results are presented in FIG. 14A and are expressed as a % of translational efficiency measured for the globin control (glo) which was set to 100%. For the 3 concentrations of RNA tested (2.7; 13.5 and 27 nM), globin expression was far above that of the PV driven constructs as expected (Borman et al., Nucleic Acids Res. 25: 925-932, 1995). Interestingly, although the translation rate of the PV construct remained low and constant at all RNA concentrations in the RRL, this was clearly not the case when the same construct was assayed in the hybrid system. In fact, at the highest RNA concentration, PV-IRES-driven translation was nearly half that of the globin renilla construct in the hybrid system (FIG. 14A) (compare with the approximately 10% reached by the PV RRL combination at highest RNA concentration). This shows that HeLa ribosomes can support PV IRES driven translation.

The next step was to conduct the reverse experiment which consists of adding ribosomes isolated from cells that do not support PV IRES translation such as Baby Hamster Kidney cells (BHK) (Borman et al., Nucleic Acids Res., 25(5): 925-932, 1997). Indeed, these cells are thought to lack one, or several, factors that are needed for PV IRES translation (Borman et al., Nucleic Acids Res., 25: 925-932, 1995; Borman et al., Nucleic Acids Res., 25(5): 925-932, 1997). Thus, this property was used to test the limits of the in vitro translation system of the invention.

In an initial control experiment, a mRNA harboring the PV IRES (or the globin 5′UTR) into BHK cells (FIG. 14B) transfected by electroporation. Analysis of the renilla activity confirmed the data of Borman and colleagues showing that PV-IRES translation was very inefficient in this cell type (Borman et al., Nucleic Acids Res., 25(5): 925-932, 1997).

Thus, a hybrid reconstituted translation system was prepared with ribosomes isolated from BHK cells that were mixed to the S100 URRL supernatant. Translation of the reporter mRNAs in the in vitro hybrid system showed that expression from the PV containing construct remained extremely low indicating that BHK ribosomes are unable to support PV-driven translation (FIG. 14C). This contrasts with the globin-renilla construct which was correctly expressed in the hybrid system indicating that the BHK ribosomes are not deficient to support global protein synthesis.

Together, these data indicate that the addition of ribosomes from a given cell type is sufficient to recreate cellular tropism for translation of IRES-driven genes.

### EXAMPLE 6

### The Hybrid System Restitutes the Effects of Cell Differentiation on Translation

The next step was to investigate whether the system of the invention could recapitulate changes in physiological conditions that can be found in cells upon different stimuli. The inventors reasoned that cell differentiation would represent an excellent model of study as during this process, the cell goes through major physiological changes that also affect protein synthesis (Ma et al., Nat. Rev. Mol. Cell. Biol., 10(5): 307-318, 2009; Thoreen et al., Nature, 485(7396): 109-113, 2012). As a first approach, the inventors looked at the expression of 3 renilla reporter genes whose translation was driven by the globin, GAPDH and the utrophin 5′UTR (which was shown to be particularly enhanced at the level of translation during myoblast differentiation (Miura et al., J. Biol. Chem. 280(38): 32997-33005, 2005). As a model for cell differentiation, C2C12 mouse myoblastic cells to which horse serum was added to induce differentiation into myotubes as previously described (Kubo, J. Physiol., 442: 743-759, 1991) were used. FIG. 15A shows the translation of capped and polyadenylated in vitro transcribed mRNAs that were electroporated in C2C12 mouse myoblastic cells. Luciferase activity was measured before (undifferentiated) and 15 h after (differentiated) horse serum addition as indicated (FIG. 15A). It can be noticed that translation from all three mRNAs was strongly stimulated in C2C12 cells that are engaged in the differentiation process. As expression from the utrophin driven construct was quite low, a blow up picture is presented below.

In order to investigate whether the effects observed in C2C12 could be recapitulated in vitro, ribosome pellets obtained from undifferentiated and differentiated C2C12 cells that were assembled with post-ribosomal supernatant from the URRL. In vitro transcribed globin, GAPDH and utrophin mRNAs described above were translated for 30 minutes before analysis of renilla activity (FIG. 15B). Interestingly, translational activity from all 3 constructs was also stimulated by the addition of ribosomes derived from differentiated C2C12 cells. Once again, this nicely shows that the reconstituted hybrid system could reproduce specific physiological conditions of living cells.

### EXAMPLE 7

### Design of a Factor-Depleted Hybrid System by the Use of RNA Silencing

In contrast with living cells, CFPS offer the possibility to manipulate the level of endogenous components by relatively simple and highly standardized biochemical protocols. As such, removal can be carried out by affinity column chromatography or immunodepletion whereas inactivation can be achieved by enzymatic cleavage, chemicals or antagonist peptides. However, there remain several limitations such as the requirement for specific antibodies or antagonists and unwanted side effects that can result from the addition of these molecules to the translation assay. Therefore, another approach was proposed. As most proteins involved in translational control are found associated with the ribosomal fraction, the inventors reasoned that efficient depletion of a given factor could be performed by the use of RNA interference in cultured cells prior to ribosome purification. To demonstrate the proof of concept of this experimental protocol, the inventors took advantage of the recent data showing that the DEAD-box RNA helicase DDX3 is required for translation of the HIV-1 genomic RNA (Soto-Rifo et al., Embo. J., 31(18): 3745-3756, 2012). For this, HeLa cells in culture were depleted from endogenous DDX3 by the use of shRNAs as described in Materials and Methods; the extent of DDX3 knockdown was checked by western blot and showed that most of the endogenous DDX3 was removed from the ribosomes (FIG. 16A). It is noteworthy that DDX3 was exclusively found on the ribosomal fraction with no detectable protein left in the post-ribosomal supernatant (FIG. 16A compare S100 with C100). Transfection of RNA constructs harboring the globin or the HIV-1 5′ UTR was then realized in HeLa cells that were treated, or not, with shRNAs against DDX3 (FIG. 16B). As previously shown (Soto-Rifo et al., Embo. J., 31(18): 3745-3756, 2012), translation from the globin-renilla RNA construct was not affected by the lack of DDX3 whereas the construct driven by the HIV-1 5′ UTR exhibited a significant drop in translational efficiency upon DDX3 knockdown (FIG. 16B).

From these DDX3 knocked-down and control HeLa cells, the ribosome fractions were isolated and added to the S100 supernatant of an untreated RRL (Su) as described above. In vitro produced RNA constructs, namely globin and HIV-1 renilla, were translated in the hybrid reconstituted system and the results are presented in FIG. 16C. Interestingly, it was observed that translation of the globin-renilla RNA was marginally affected whereas translation from the HIV-1 5′ UTR containing mRNA was severely reduced (FIG. 16C) in the hybrid system that was assembled with ribosomes that lack DDX3. Once again, these results are matching data obtained in living cells (FIG. 16B) showing that the in vitro system faithfully recapitulates the physiological conditions of the cell. It is noteworthy that the level of inhibition upon DDX3 depletion was higher in the in vitro system than in cells.

### EXAMPLE 8

### Use of the Hybrid System from a cDNA Transfection

As the hybrid system relies on the isolation of crude ribosomes from cells, it was proposed to take advantage of this situation to isolate ribosome associated RNAs from an ectopically expressed cDNA plasmid. As such, the ectopic gene would be synthesized, processed and exported by the cellular machinery; it would undergo transcription, splicing, capping/polyadenylation, nucleus export and translation (see cartoon FIG. 17A). Once translated in the cytoplasm, the ectopic RNA must be associated to polysomes and should be co-purified with the ribosomal fraction.

To verify this, different concentrations of the cDNA plasmid coding for β-globin-renilla into HeLa cells in culture were transfected. 48 hours after transfection, cytoplasmic RNA concentration was measured by quantitative RT-PCR and data are summarized in FIG. 17B. This shows a correlation between the amount of plasmid transfected (from 0.5; 6 and 12 μg of input cDNA per 1.5·10⁷ cells) and the concentration of neo-synthesized renilla-globin RNA found in the cytoplasm (FIG. 17B; from 0.010 picogrammes to 0.190 pg of RNA).

Then, the hybrid system was assembled by mixing these ribosome containing RNAs to the supernatant obtained from fractionation of the URRL. Upon reconstitution, the hybrid system was incubated for 30 minutes at 30° C. before quantification of luciferase activity (FIG. 17C). It can be observed that the production of luciferase correlated with the amount of RNAs quantified by quantitative RT-PCR. In order to rule out any possible interference with some luciferase protein that could have been co-purified with the HeLa ribosomes, cycloheximide was added to the reconstituted system and this gave virtually no enzymatic activity under these experimental conditions (data not shown). This shows that the RNAs trapped into cellular polysomes are fully functional for translation upon their transfer to the hybrid reconstituted system.

This is a particularly attractive alternative to in vitro transcription as it ensures that the RNA of interest has been produced in its native environment (and as such, could be spliced) and has undergone the pioneer round of translation in the cytoplasm: these two steps being involved in the overall efficiency at which a mRNA is translated (Bedard et al., Embo. J., 26(2): 459-467, 2007; Maquat et al., Cell 142(3): 368-374, 2010; Sanford et al., Genes Dev., 18(7): 755-768, 2004).

To investigate the impact of nuclear genesis of an ectopic mRNA on translation, the c-myc IRES as a model study was used. Expression from this IRES has been reported to be very inefficient in in vitro systems (Stoneley et al., Nucleic Acids Res., 28(3): 687-694, 2000b) and this was explained by a need for this IRES to be transcribed and folded within the nucleus in order to acquire specific IRES Trans Acting Factors (ITAFs) that are needed for its activation (Stoneley et al., Oncogene, 23(18): 3200-3207, 2004).

Thus, the c-myc 5′UTR was a good tool to test the in vitro translation system of the invention. A capped and polydenylated c-myc driven renilla reporter RNA together with the globin-renilla control was first generated. Both were translated in the RRL (FIG. 18A) and it can be observed that production of luciferase under the control of the c-myc 5′UTR was about 10-fold lower than that from globin in agreement with the work of Willis and colleagues (Stoneley et al., Mol. Cell. Biol. 20(4): 1162-1169, 2000a). This sharply contrasts with expression from the corresponding plasmid cDNAs in HeLa cells which show a very good level of translation from the c-myc driven construct as previously described (Stoneley et al., Nucleic Acids Res., 28(3): 687-694, 2000b). Then, ribosomes were isolated from the cDNA transfected HeLa cells to supplement the supernatant obtained from URRL just as previously described (see above). The reconstituted system was incubated for 30 minutes at 30° C. before analysis of luciferase production (FIG. 18C). Interestingly, this showed that the c-myc derived constructs that co-sedimented with the HeLa ribosome pellet were efficient in the hybrid reconstituted system. In fact, the ratio between translational efficiency of globin/c-myc was the same than that observed in HeLa cells upon cDNA transfection (FIG. 18B). This confirms the adaptability of the hybrid translation system to recapitulate translational properties encountered in cultured cells.

The ability of the hybrid system to produce proteins from endogenous mRNAs associated to ribosomes makes it a valuable tool for large scale analyses.

Indeed, cellular endogenous mRNAs that are associated to translating ribosomes can then be extracted from the ribosomal pellet and subjected to high throughput RNA sequencing as described for example in Roberts et al. (2011) Genome Med. 3:74 or Wilhelm et al. (2008) Nature 453:1239-1243. These mRNAs are indeed representative of the whole transcriptome that is being translated under the experimental conditions used.

These cellular endogenous mRNAs can also be transferred in the hybrid system of the invention, thereby producing detectable amounts of proteins that can then be detected, identified and quantified in particular by mass spectrometry. To facilitate this detection, identification and quantification by mass spectrometry, the amino acid mix added in the in vitro hybrid system is replaced by “heavy” amino acids to detect specifically newly synthetized proteins, as described for example in Bantscheff et al. (2007) Anal. Bioanal. Chem. 389:1017-1031. Alternatively, the in vitro hybrid system is supplied with biotinylated puromycin molecules that are incorporated into neo-synthesized peptides and enable purification of these peptides with streptavidin, as described for example in Aviner et al. (2013) Genes Dev. 27:1834-1844.

### EXAMPLE 9

### Use of the Hybrid System to Label all the Newly Synthetized Protein (Cellular and Viral) in the Case of Viral Infection

As an extension of Example 8, isolation of ribosomes from cells that were previously infected with viruses also contained viral mRNAs. Once transferred in the hybrid system described in Example 8, these viral mRNAs were translated to yield viral proteins that could be detected by radiolabeled methionine incorporation and could be further quantified and purified.

FIG. 20 shows such ribosomes isolated from A549 cells infected with influenza virus. The neo-synthetized viral proteins can clearly be observed and are labeled on the right side of the picture.

1. A method for translating in vitro a ribonucleic acid template into an amino acid sequence of interest, the method using a translation reaction mixture comprising: (i) a ribosome-depleted red blood cells lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

2. The method according to claim 1, said method comprising:
pre-a) optionally preparing a translation reaction mixture comprising:
(i) a ribosome-depleted red blood cells lysate,
(ii) ribosomes isolated from eukaryotic cells,
with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes;
a) to form a translation system, contacting the translation reaction mixture as defined in step (pre-a) with:
(i) the ribonucleic acid template (RNA); or
(ii) a transcription reaction mixture comprising
(i) a DNA encoding the amino acid sequence of interest, and
(ii) the necessary element for transcribing said DNA into the ribonucleic acid template;
and
b) incubating the translation system of (a) for a time sufficient to achieve translation of the ribonucleic acid template into the amino acid sequence of interest.

3. The method according to claim 1, said method comprising:
pre-a) transfecting a DNA encoding the amino acid sequence of interest into eukaryotic cells, with proviso that the eukaryotic cells are not reticulocytes;
a) after a time sufficient to allow the eukaryotic transfected cells to achieve RNA transcription and to begin translation, isolating ribosomes from the transfected cells of step (pre-a) under conditions that allow the RNA produced from the DNA encoding the amino acid sequence of interest to be isolated together with the ribosomes; and
b) incubating a translation system comprising (i) a ribosome-depleted red blood cells lysate, (ii) ribosomes isolated from the eukaryotic cells in step (a) for a time sufficient to achieve translation of the ribonucleic acid template into the amino acid sequence of interest.

4. The method according to claim 2, wherein step (b) is conducted during at least 15 minutes.

5-6. (canceled)

7. A cell-free translation reaction system for translating in vitro a RNA into a protein comprising: (i) a ribosome-depleted reticulocyte lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

8. An in vitro method for analyzing an actively translated transcriptome of eukaryotic cells of interest comprising:
a) isolating ribosomes from eukaryotic cells of interest under conditions that allow RNAs produced in the eukaryotic cells of interest to be isolated together with the ribosomes;
b) incubating a translation system comprising (i) a ribosome-depleted red blood cells lysate and (ii) ribosomes and RNAs isolated from the eukaryotic cells of interest in step a), for a time sufficient to achieve translation of the RNAs, isolated with the ribosomes in step a), into the corresponding amino acid sequences,
with the proviso that (1) when the ribosome-depleted red blood cell lysate is obtained from a nuclease untreated rabbit reticulocyte lysate, the eukaryotic cells of interest from which ribosomes are isolated are not nuclease rabbit untreated reticulocytes, and (2) when the ribosome-depleted red blood cell lysate is obtained from a nuclease treated rabbit reticulocyte lysate, the eukaryotic cells of interest from which ribosomes are isolated are not nuclease treated rabbit reticulocytes; and
c) identifying and optionally quantifying the amino acid sequences obtained in step b).

9. A kit for translating in vitro a ribonucleic acid template into an amino acid sequence of interest comprising, in separate containers or in the same container, (i) a ribosome-depleted reticulocyte lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

10. A kit for producing a translation reaction mixture comprising, in separate containers or in the same container, (i) a ribosome-depleted reticulocyte lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

11. A kit according to claim 9, wherein said kit further comprises, in separate containers or in the same container, at least one element chosen from the group consisting of:
at least a tRNA;
at least an aminoacyl-tRNA synthetase;
at least an initiation factor;
at least an elongation factors;
at least a termination factor;
at least a chaperone;
at least a foldase;
at least an amino acid;
at least a labelled amino acid, for instance a radiolabelled amino acid;
at least an energy source;
at least an energy regenerating system;
salts;
a buffer solution.

12. A kit for analyzing an actively translated transcriptome of eukaryotic cells of interest comprising, in separate containers or in the same container:
a ribosome-depleted reticulocyte lysate, and
at least one element chosen from the group consisting of:
at least a labelled amino acid,
at least a biotinylated puromycin, and
at least an immobilized streptavidin.

13. The method according to claim 1, wherein the eukaryotic cells from which ribosomes (ii) are isolated are not reticulocytes.

14. The method according to claim 1, wherein the ribosome-depleted red blood cell lysate (i) is obtained from rabbit red blood cells.

15. The method according to claim 1, wherein the eukaryotic cells from which ribosomes (ii) are isolated are human cells.

16. The method according to claim 1, wherein the ribosome-depleted red blood cell lysate (i) is obtained from a red blood cell lysate treated with a nuclease.

17. The method according to claim 1, wherein the ribosome-depleted red blood cell lysate (i) is obtained from rabbit reticulocytes treated with a nuclease, and the eukaryotic cells from which ribosomes (ii) are isolated are human cells.

18-22. (canceled)

23. The kit according to claim 10, wherein the eukaryotic cells from which ribosomes (ii) are isolated are not reticulocytes.

24. The kit according to claim 10, wherein the ribosome-depleted red blood cell lysate (i) is obtained from rabbit red blood cells.

25. The kit according to claim 10, wherein the eukaryotic cells from which ribosomes (ii) are isolated are human cells.

26. The kit according to claim 10, wherein the ribosome-depleted red blood cell lysate (i) is obtained from a red blood cell lysate treated with a nuclease.

27. The kit according to claim 10, wherein the ribosome-depleted red blood cell lysate (i) is obtained from rabbit reticulocytes treated with a nuclease, and the eukaryotic cells from which ribosomes (ii) are isolated are human cells.

28. A method for in vitro producing a peptide, comprising the use of a translation reaction mixture comprising (i) a ribosome-depleted red blood cell lysate, (ii) ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

29. A method for producing translation reaction mixture comprising the use of a ribosome-depleted reticulocyte lysate and ribosomes isolated from eukaryotic cells, with the proviso that (1) when the ribosome-depleted red blood cell lysate is obtained from a nuclease untreated rabbit reticulocyte lysate, the eukaryotic cells from which ribosomes are isolated are not nuclease untreated rabbit reticulocytes, and (2) when the ribosome-depleted red blood cell lysate is obtained from a nuclease treated rabbit reticulocyte lysate, the eukaryotic cells from which ribosomes are isolated are not nuclease treated rabbit reticulocytes.

30. The method according to claim 3, wherein step (b) is conducted during at least 15 minutes.

31. The method according to claim 2, wherein the eukaryotic cells from which ribosomes (ii) are isolated are not reticulocytes.

32. The method according to claim 3, wherein the eukaryotic cells from which ribosomes (ii) are isolated are not reticulocytes.

33. The method according to claim 2, wherein the ribosome-depleted red blood cell lysate (i) is obtained from rabbit red blood cells.

34. The method according to claim 3, wherein the ribosome-depleted red blood cell lysate (i) is obtained from rabbit red blood cells.

35. The method according to claim 2, wherein the eukaryotic cells from which ribosomes (ii) are isolated are human cells.

36. The method according to claim 3, wherein the eukaryotic cells from which ribosomes (ii) are isolated are human cells.

37. The method according to claim 2, wherein the ribosome-depleted red blood cell lysate (i) is obtained from a red blood cell lysate treated with a nuclease.

38. The method according to claim 3, wherein the ribosome-depleted red blood cell lysate (i) is obtained from a red blood cell lysate treated with a nuclease.

39. The method according to claim 2, wherein the ribosome-depleted red blood cell lysate (i) is obtained from rabbit reticulocytes treated with a nuclease, and the eukaryotic cells from which ribosomes (ii) are isolated are human cells.

40. The method according to claim 3, wherein the ribosome-depleted red blood cell lysate (i) is obtained from rabbit reticulocytes treated with a nuclease, and the eukaryotic cells from which ribosomes (ii) are isolated are human cells.


SEQUENCE LISTING (2 sequences)

SEQ ID NO: 1 - DNA, 47 bp, Artificial Sequence
  Synthetic shCtrl
gcagcacgacttcttcaagttcaagagacttgaagaagtcgtgctgc

SEQ ID NO: 2 - DNA, 49 bp, Artificial Sequence
  Synthetic shDDX3
gatgctggctcgtgatttctttcaagagaagaaatcacgagccagcatc